(19) United States
(12) Patent Application Publication
(10) Pub. No.: US 2023/0405153 A1
(43) Pub. Date: Dec. 21, 2023

(54) FIBROBLAST ACTIVATION PROTEIN-TARGETED MAGNETIC RESONANCE IMAGING AGENTS, COMPOSITIONS, AND METHODS OF USE

(71) Applicant: Purdue Research Foundation, West Lafayette, IN (US)

(72) Inventors: Philip Stewart Low, West Lafayette, IN (US); Ramesh Mukkamala, West Lafayette, IN (US); Madduri Srinivasarao, West Lafayette, IN (US)

(21) Appl. No.: 18/336,708

(22) Filed: Jun. 16, 2023

Related U.S. Application Data
(60) Provisional application No. 63/353,042, filed on Jun. 17, 2022.

(51) Int. Cl. A61K 49/08; A61K 49/10

(52) U.S. Cl. CPC A61K 49/085; A61K 49/108

(57) ABSTRACT

A conjugate comprising FL-L-IA, wherein FL is a radical of a small molecule ligand that specifically binds with fibroblast activation protein (FAP), L is a linker, which binds an FL to IA, and IA is a radical of a magnetic resonance imaging (MRI) agent, or a pharmaceutically acceptable salt thereof; a composition comprising same; and a method of using the conjugate or composition to image cells, a tissue, or an organ that express(es) FAP with magnetic resonance imaging.

### CROSS-REFERENCE TO RELATED APPLICATIONS

This application claims the benefit of U.S. Provisional Application No. 63/353,042, filed Jun. 17, 2022, which is incorporated by reference as if fully set forth herein.

### TECHNICAL FIELD

The present disclosure relates to fibroblast activation protein (FAP), conjugates comprising same and magnetic resonance imaging (MRI) agents, compositions comprising such conjugates, and methods of using the conjugates and the compositions.

### BACKGROUND

Magnetic resonance imaging (MRI) agents are used to image various cancers and injuries to soft tissues, joints, the brain, the spinal cord, the heart, the digestive organs, and the like. MRI agents currently in use, however, are not targeted and, therefore, are not specific in their uptake. Non-specificity of uptake leads to reduced contrast, due to high background signal, and increased toxicity.

In view of the above, there remains a need for MRI agents that overcome the disadvantages of reduced contrast and increased toxicity of non-targeted MRI agents. Accordingly, it is an object of the present disclosure to provide targeted MRI agents. This and other object and advantages, as well as inventive features, will be apparent from the detailed description provided herein.

### SUMMARY

Provided is a conjugate comprising FL-L-IA. FL is a radical of a small molecule ligand that specifically binds with fibroblast activation protein (FAP), L is a linker, which binds an FL to IA, and IA is a radical of a magnetic resonance imaging (MRI) agent, or a pharmaceutically acceptable salt thereof.

FL can have a structure of formula I:

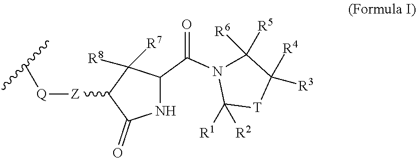

wherein Q is aryl, heteroaryl, or heterocyclyl;
Z is a bond, substituted or unsubstituted C₁-C₃ alkylene, substituted or unsubstituted heteroalkylene, amino, —O—, or —S—;
T is substituted or unsubstituted methylene, substituted or unsubstituted amino, —O—, or —S—;
R¹ and R² are each independently selected from the group consisting of —H, —CN, —CHO, —B(OH)₂, —C(O)alkyl, —C(O)aryl-, —OC—C(O)aryl, —C═C—S(O)₂ aryl, —CO₂H, —SO₃H, —SO₂NH₂, —PO₃H₂, —SO₂F, —CONH₂, and 5-tetrazolyl;
R³ and R⁴ are each independently selected from the group consisting of —H, —OH, F, Q, Br, I, —C₁₋₆, alkyl, —O—C₁₋₆ alkyl, and —S—C₁₋₆alkyl; and
R⁵, R⁶, R⁷, and R⁸ are each independently selected from the group consisting of H, alkyl, and halo. Alternatively, FL can have a structure of formula II:

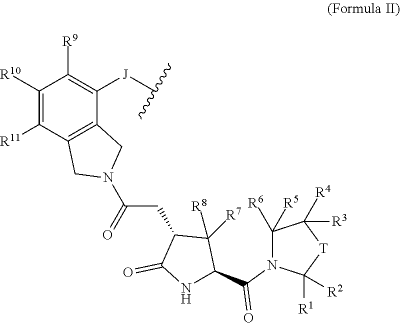

wherein:
T is substituted or unsubstituted methylene, substituted or unsubstituted amino, —O—, or —S—;
J is C(R^{J})₂, wherein each R^{J} is independently H or alkyl, or both R^{J}, when taken together, form oxo;
R¹ and R² are each independently selected from the group consisting of —H, —CN, —CHO, —B(OH)₂, —C(O)alkyl, —C(O)aryl-, —C═C—C(O)aryl, —C—S(O)₂aryl, —CO₂H, —SO₃H, —SO₂NH₂, —PO₃H₂, —SO₂F, —CONH₂, and 5-tetrazolyl;
R³ and R⁴ are each independently selected from the group consisting of H, —OH, F, Q, Br, I, —C₁₋₆ alkyl, —O—C₁₋₆ alkyl, and —S—C₁₋₆ alkyl, wherein Q is aryl, heteroaryl, or heterocyclyl;
R⁵, R⁶, R⁷, and R⁸ are each independently selected from group consisting of H, alkyl, and halo; and
R⁹, R¹⁰, and R¹¹ are each independently selected from group consisting of H, —C₁₋₆ alkyl, —C₁₋₆ haloalkyl, —O—C₁₋₆ alkyl, —S—C₁₋₆ alkyl, F, Q, Cl, Br and I.
FL can have a structure selected from the group consisting of:

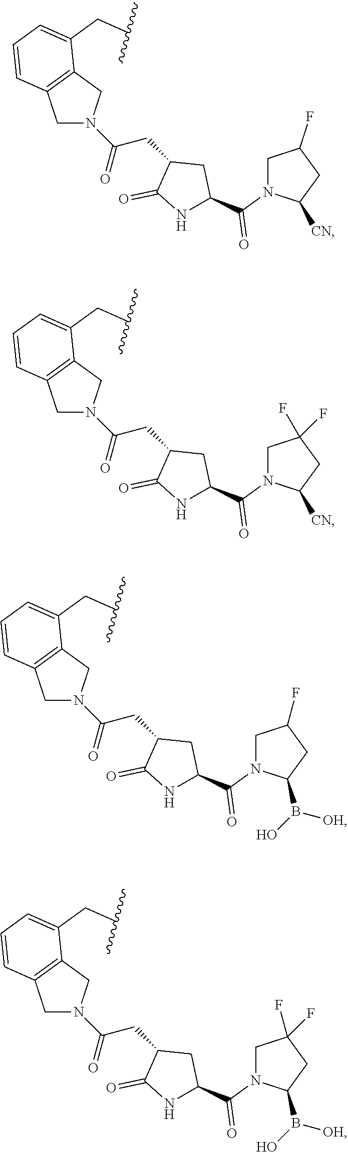 [SMILES: *Cc1cccc2c1CN(C(=O)C[C@@H]1C[C@@H](C(=O)N3CC(F)C[C@H]3C#N)NC1=O)C2 ; *Cc1cccc2c1CN(C(=O)C[C@@H]1C[C@@H](C(=O)N3CC(F)(F)C[C@H]3C#N)NC1=O)C2 ; *Cc1cccc2c1CN(C(=O)C[C@@H]1C[C@@H](C(=O)N3CC(F)C[C@H]3B(O)O)NC1=O)C2 ; *Cc1cccc2c1CN(C(=O)C[C@@H]1C[C@@H](C(=O)N3CC(F)(F)C[C@H]3B(O)O)NC1=O)C2]

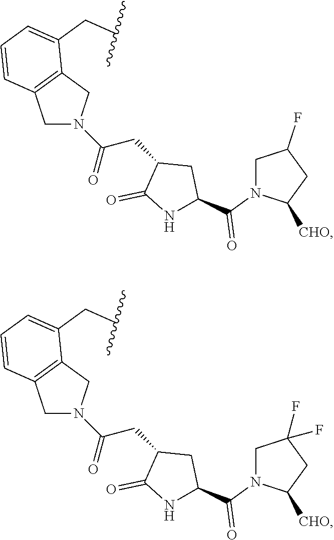 [SMILES: *Cc1cccc2c1CN(C(=O)C[C@@H]1C[C@@H](C(=O)N3CC(F)C[C@H]3C=O)NC1=O)C2 ; *Cc1cccc2c1CN(C(=O)C[C@@H]1C[C@@H](C(=O)N3CC(F)(F)C[C@H]3C=O)NC1=O)C2]

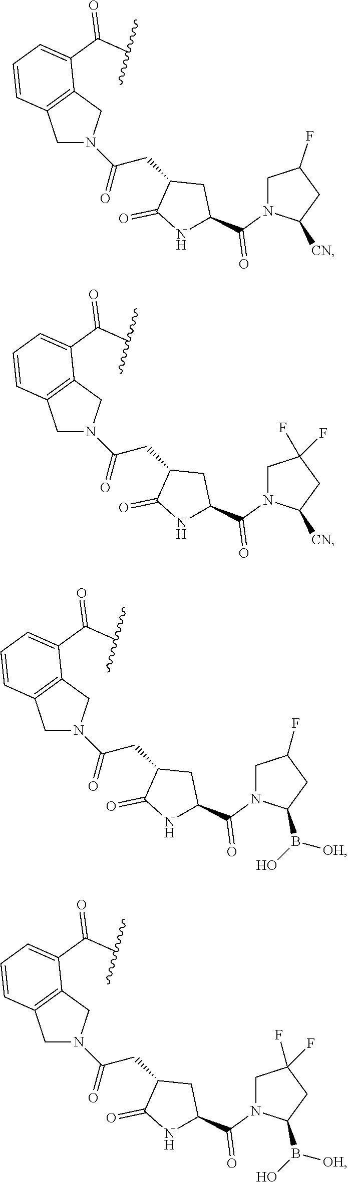 [SMILES: *C(=O)c1cccc2c1CN(C(=O)C[C@@H]1C[C@@H](C(=O)N3CC(F)C[C@H]3C#N)NC1=O)C2 ; *C(=O)c1cccc2c1CN(C(=O)C[C@@H]1C[C@@H](C(=O)N3CC(F)(F)C[C@H]3C#N)NC1=O)C2 ; *C(=O)c1cccc2c1CN(C(=O)C[C@@H]1C[C@@H](C(=O)N3CC(F)C[C@H]3B(O)O)NC1=O)C2 ; *C(=O)c1cccc2c1CN(C(=O)C[C@@H]1C[C@@H](C(=O)N3CC(F)(F)C[C@H]3B(O)O)NC1=O)C2]

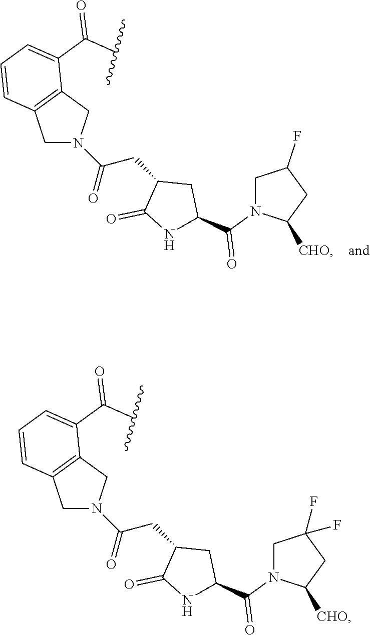 [SMILES: *C(=O)c1cccc2c1CN(C(=O)C[C@@H]1C[C@@H](C(=O)N3CC(F)C[C@H]3C=O)NC1=O)C2 ; "and" ; *C(=O)c1cccc2c1CN(C(=O)C[C@@H]1C[C@@H](C(=O)N3CC(F)(F)C[C@H]3C=O)NC1=O)C2]

Alternatively, FL can have a structure selected from the group consisting of:

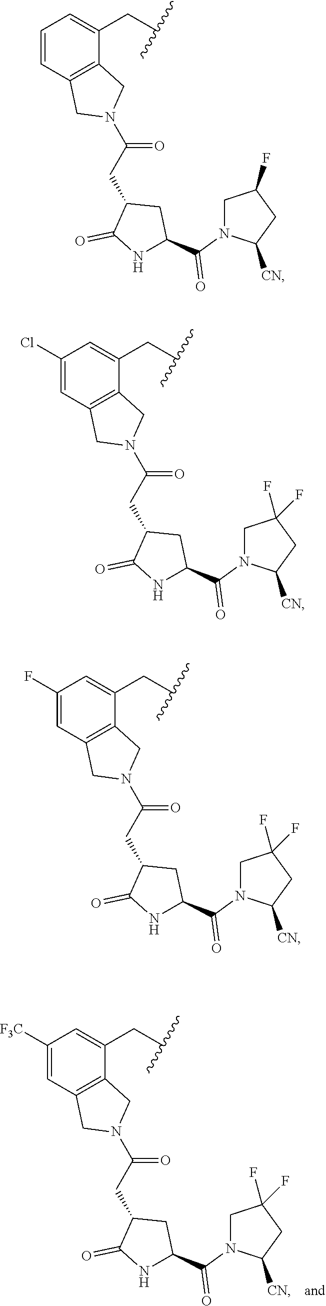 [SMILES: *Cc1cccc2c1CN(C(=O)C[C@@H]1C[C@@H](C(=O)N3C[C@@H](F)C[C@H]3C#N)NC1=O)C2 ; *Cc1cc(Cl)cc2c1CN(C(=O)C[C@@H]1C[C@@H](C(=O)N3CC(F)(F)C[C@H]3C#N)NC1=O)C2 ; *Cc1cc(F)cc2c1CN(C(=O)C[C@@H]1C[C@@H](C(=O)N3CC(F)(F)C[C@H]3C#N)NC1=O)C2 ; *Cc1cc(C(F)(F)F)cc2c1CN(C(=O)C[C@@H]1C[C@@H](C(=O)N3CC(F)(F)C[C@H]3C#N)NC1=O)C2]

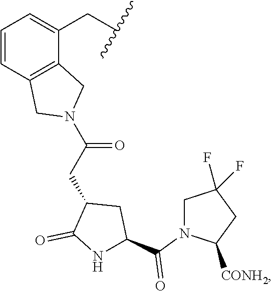 [SMILES: *Cc1cccc2c1CN(C(=O)C[C@@H]1C[C@@H](C(=O)N3CC(F)(F)C[C@H]3C(N)=O)NC1=O)C2]

L can comprise one or more linker groups independently selected from the group consisting of alkyl(ene), heteroalkyl(ene), heterocycloalkyl(ene), heteroaryl, aryl, alkoxy, thioether, disulfide, carboxylic acid, anhydride, carbonate, carbamate, thioether, sugar, and peptide. L can comprise one or more linker groups independently selected from the group consisting of polyethylene glycol (PEG), alkyl(ene), amide, carboxylic acid, anhydride, carbonate, ester, carbamate, thioether, phenyl, and triazole.

The IA can be (CM), in which M is a metal (e.g., gadolinium (Gd³⁺), manganese (Mn²⁺), or dysprosium (Dy³⁺) and C is a chelator. C can be selected from the group consisting of DOTA (1,4,7,10-tetraazacyclododecane-1,4,7,10-tetraacetic acid), TETA (1,4,8,11-tetraazacyclotetradecane-1,4,8,11-tetraacetic acid), SarAr (1-N-(4-aminobenzyl)-3,6,10,13,16,19-hexaacabicyclo[6.6.6]-eicosane-1,8-diamine), NOTA (1,4,7-triazacyclononane-1,4,7-triacetic acid), NETA (4-[2-(bis-carboxymethylamino)-ethyl]-7-carboxymethyl-[1,4,7]triazonan-1-yl) acetic acid, DTPA (diethylenetriaminepentaacetic acid), or a derivative of any of the foregoing.

C can have a structure selected from:

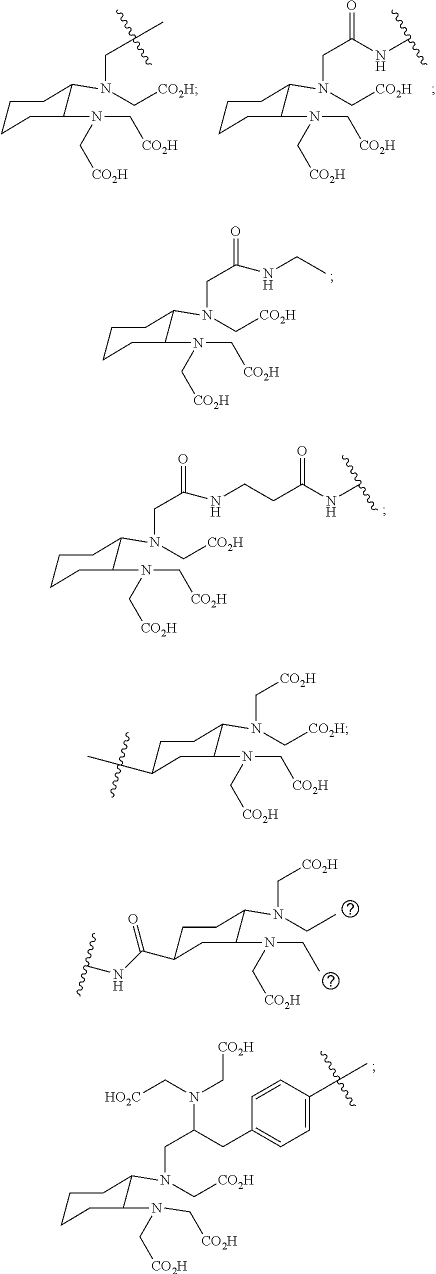

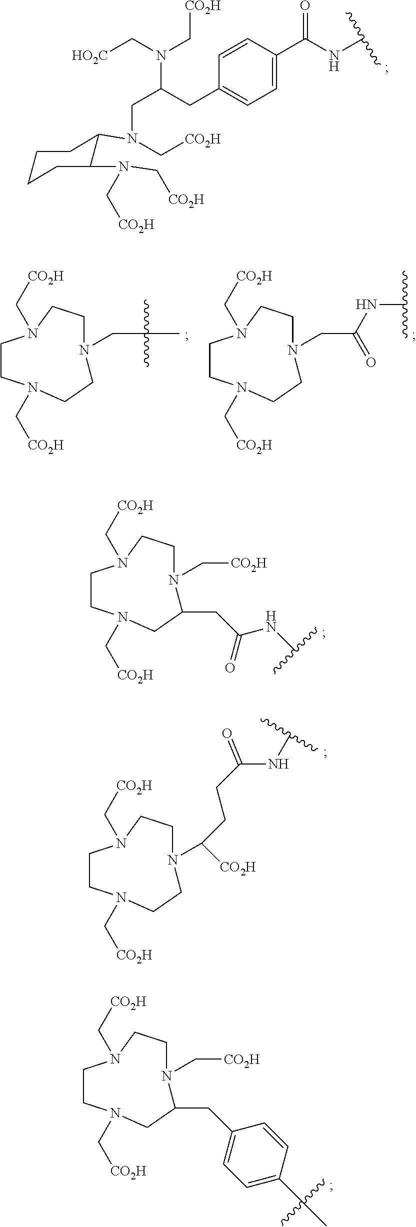 [SMILES: *NC(=O)c1ccc(CC(CN(CC(=O)O)C2CCCCC2N(CC(=O)O)CC(=O)O)N(CC(=O)O)CC(=O)O)cc1 ; *CN1CCN(CC(=O)O)CCN(CC(=O)O)CC1 ; *NC(=O)CN1CCN(CC(=O)O)CCN(CC(=O)O)CC1 ; *NC(=O)CC1CN(CC(=O)O)CCN(CC(=O)O)CCN1CC(=O)O ; *NC(=O)CCC(C(=O)O)N1CCN(CC(=O)O)CCN(CC(=O)O)CC1 ; *c1ccc(CC2CN(CC(=O)O)CCN(CC(=O)O)CCN2CC(=O)O)cc1]

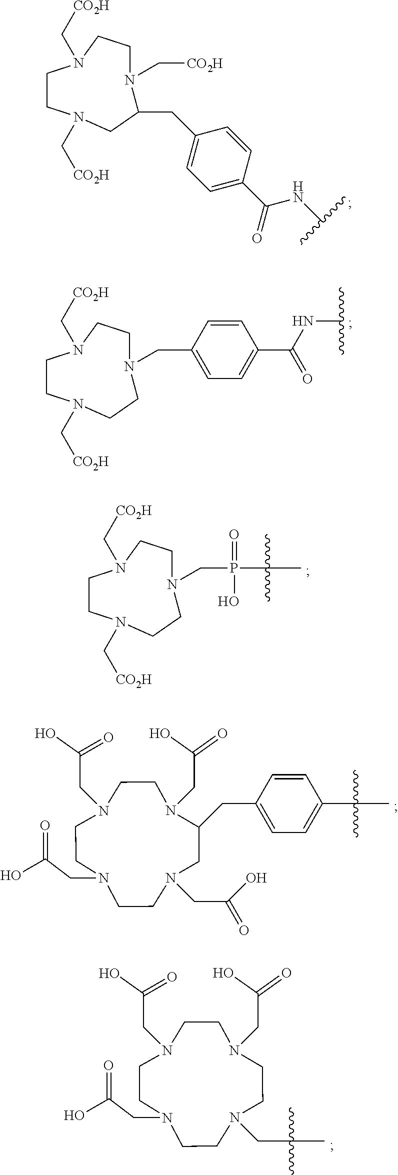

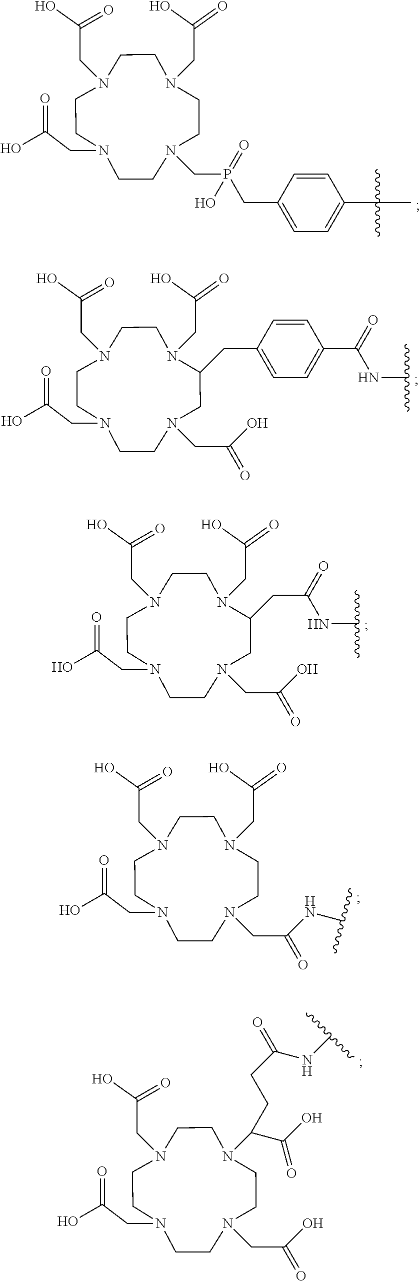 [SMILES: *c1ccc(CP(=O)(O)CN2CCN(CC(=O)O)CCN(CC(=O)O)CCN(CC(=O)O)CC2)cc1 ; *NC(=O)c1ccc(CC2CN(CC(=O)O)CCN(CC(=O)O)CCN(CC(=O)O)CCN2CC(=O)O)cc1 ; *NC(=O)CC1CN(CC(=O)O)CCN(CC(=O)O)CCN(CC(=O)O)CCN1CC(=O)O ; *NC(=O)CN1CCN(CC(=O)O)CCN(CC(=O)O)CCN(CC(=O)O)CC1 ; *NC(=O)CCC(C(=O)O)N1CCN(CC(=O)O)CCN(CC(=O)O)CCN(CC(=O)O)CC1]

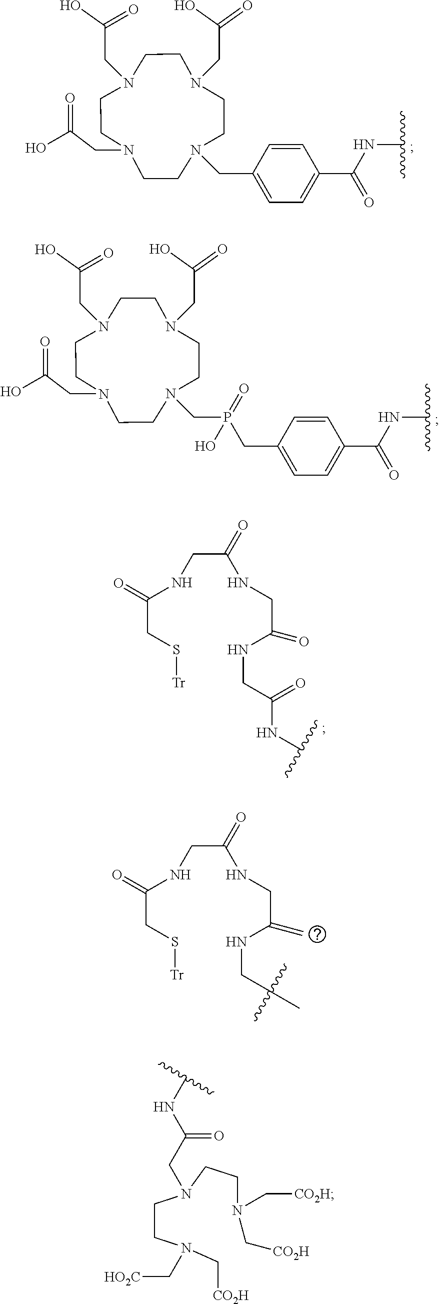

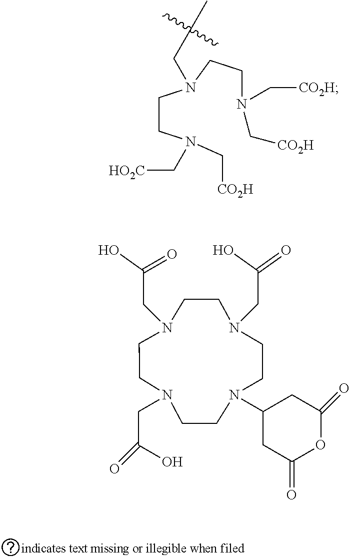 [SMILES: *CN(CCN(CC(=O)O)CC(=O)O)CCN(CC(=O)O)CC(=O)O ; O=C(O)CN1CCN(CC(=O)O)CCN(C2CC(=O)OC(=O)C2)CCN(CC(=O)O)CC1]

The MRI agent can be a paramagnetic contrast agent. The paramagnetic contrast agent can comprise gadolinium (Gd³⁺), manganese (Mn²⁺), or dysprosium (Dy³⁺). The paramagnetic contrast agent can be Gd-IOTA (gadoterate dotarem), Gd-HP-DO3A (gadoteridol), Gd-BT-DO3A (gadobutrol), Gd-DTPA (gadopentate dimeglumine), Gd-DTPA-BMEA (gadoversetamide), Gd-DTPA-BMA (gadodiamide), Gd-BOPTA (gadobenate dimeglumine), Gd-EOB-DTPA (gadoxetate), or Ms-325 (gadofosveset).

Further provided is a pharmaceutical composition. The pharmaceutical composition comprises an above-described conjugate and a pharmaceutically acceptable carrier.

Still further provided is a method of imaging cells, a tissue, or an organ, any of which express(es) FAP, in a subject. The method comprises administering to the subject an above-described conjugate or a pharmaceutical composition comprising the conjugate and a pharmaceutically acceptable carrier, and having the cells, the tissue, or the organ in the subject imaged with magnetic resonance imaging.

### DETAILED DESCRIPTION

### Conjugates

Provided is a conjugate comprising FL-L-IA. FL is a radical of a small molecule ligand that specifically binds with fibroblast activation protein (FAP), L is a linker, which binds an FL to IA, and IA is a radical of a magnetic resonance imaging (MRI) agent, or a pharmaceutically acceptable salt thereof.

FL can be any suitable radical of any suitable small molecule ligand that specifically binds with FAP, such as FAPα, e.g., a PAP expressed in cancer, fibrosis, or any other disease, disorder, or condition in which FAP is expressed or overexpressed. FL can have a molecular weight below 10,000. FL can have a structure of

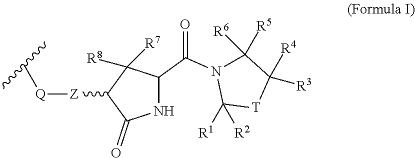

wherein Q is aryl, heteroaryl, or heterocyclyl;
Z is a bond, substituted or unsubstituted C₁-C₃ alkylene, substituted or unsubstituted heteroalkylene, amino, —O—, or —S—;
T is substituted or unsubstituted methylene, substituted or unsubstituted amino, —O—, or —S—;
R¹ and R² are each independently selected from the group consisting of —H, —CN, —CHO, —B(OH)₂, —C(O)alkyl, —C(O)aryl-, —OC—C(O)aryl, —C═C—C(O)aryl, —C═C—S(O)₂ aryl, —CO₂H, —SO₃H, —SO₂NH₂, —PO₃H₂, —SO₂F, —CONH₂, and 5-tetrazolyl;
R³ and R⁴ are each independently selected from the group consisting of —H, —OH, F, Q, Cl, Br, I, —C₁₋₆ alkyl, —O—C₁₋₆ alkyl, and —S—C₁₋₆ alkyl; and
R⁵, R⁶, R⁷, and R⁸ are each independently selected from the group consisting of H, alkyl, and halo.

The heterocyclyl of Q can comprise an aryl or a non-aryl ring structure, such as a 5- to 10-membered N-containing aromatic or non-aromatic, mono- or bicyclic heterocycle, which can optionally further comprise 1-3 heteroatoms selected from O, N and S. Q can be attached to L at a heteroalkyl, an alkyl, or an aryl position of Q. Q can be attached to L at an aryl position of Q. Q can be attached to L via a nitrogen atom (e.g., of L). Q can be attached to L via a triazolyl or an amide (e.g., of L). The heteroaryl can comprise aryl and non-aryl ring structures. The heteroaryl or the heterocyclyl can comprise 1 to 3 heteroatoms selected from O, N, and S. The heterocyclyl can comprise 1 to 3 heteroatoms selected from O, N, and S. Q can be a 5- to 10-membered N-containing aromatic or non-aromatic mono- or bicyclic heterocycle (e.g., optionally comprising aryl and non-aryl ring structures). Q can be a N-attached heterocyclyl (e.g., optionally comprising aryl and non-aryl ring structures). Q can be a C₆-C₉—N-attached heterocyclyl (e.g., optionally comprising aryl and non-aryl ring structures). The N-attached heterocyclyl can be attached to Z via a N-heterocycloalkyl. Q can be (e.g., an N-attached) isoindolinyl (e.g., wherein the N is attached to Z).

Z can be unsubstituted methylene, substituted methylene, unsubstituted ethylene, or substituted ethylene, such as ethylene substituted with oxo. The substituted or unsubstituted heteroalkylene for Z can be 1-3 atoms in length. Z can be —C(CO)—CH₂—.

T can be substituted with C₁-C₃ alkyl, haloalkyl, or halo.

Alternatively, FL can have a structure of formula II:

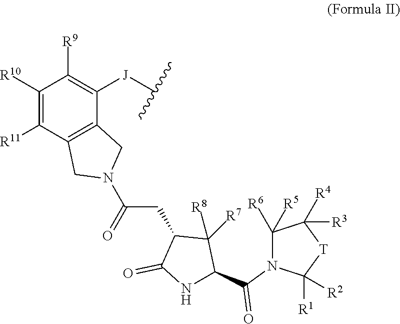

wherein:
T is substituted or unsubstituted methylene, substituted or unsubstituted amino, —O—, or —S—;
J is C(R^{J})₂, wherein each R^{J} is independently H or alkyl, or both R^{J}, when taken together, form oxo;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, —CONH₂, and 5-tetrazolyl;
R³ and R⁴ are each independently selected from the group consisting of H, —OH, F, Q, Cl, Br, I, —C₁₋₆ alkyl, —O—C₁₋₆ alkyl, and —S—C₁₋₆alkyl, wherein Q is aryl, heteroaryl, or heterocyclyl;
R⁵, R⁶, R⁷, and R⁸ are each independently selected from group consisting of H, alkyl, and halo; and
R⁹, R¹⁰, and R¹¹ are each independently selected from group consisting of H, C₁₋₆ alkyl, —C₁₋₆ haloalkyl, —O—C₁₋₆ alkyl, —S—C₁₋₆ alkyl, F, Q, Cl, Br and I.

The heterocyclyl of Q can comprise an aryl or a non-aryl ring structure, such as a 5- to 10-membered N-containing aromatic or non-aromatic, mono- or bicyclic heterocycle, which can optionally further comprise 1-3 heteroatoms selected from O, N and S.

T can be substituted with C₁-C₃ alkyl, C₁-C₃ haloalkyl, or halo (e.g., such as when T is methylene).

J can be attached to L. J can be attached to L via a nitrogen atom. J can be attached to L via a triazolyl or an amide (e.g., of L). J can be C₁-C₃ alkyl. J can be —CH₂—. J can be —CH₂CH₂—. J can be C═O.

R¹ and R² can each be independently selected from the group consisting of H, —CN, —CHO, and —B(OH)₂. R¹ and R² can each be independently selected from the group consisting of H, —CN, —CHO, and —CONH₂. R¹ can be H. R² can be —CN, —CHO, —B(OH)₂, or —CONH₂. R¹ can be H and R² can be —CN, —CHO, —B(OH)₂, or —CONH₂. R¹ can be H and R² can be —CN. R¹ can be H and R² can be —CHO. R¹ can be H and R² can be —B(OH)₂. R¹ can be H and R² can be —CONH₂.

R³ and R⁴ can each be independently —H or —F. R³ can be H and R⁴ can be —F. R³ can be F and R⁴ can be —F.

R¹ can be H, R² can be —CN, R³ can be H and R⁴ can be —F. R¹ can be H, R² can be —CN, R³ can be F and R⁴ can be —F. R¹ can be H, R² can be —CHO, R³ can be H and R⁴ can be —F. R¹ can be H, R² can be —CHO, R³ can be F and R⁴ can be —F. R¹ can be H, R² can be —B(OH)₂, R³ can be H and R⁴ can be —F. R¹ can be H, R² can be —B(OH)₂, R³ can be F and R⁴ can be —F. R¹ can be H, R² can be —CONH₂, R³ can be H and R⁴ can be —F. R¹ can be H, R² can be —CONH₂, R³ can be F and R⁴ can be —F.

R⁵, R⁶, R⁷, and R⁸ can each be H.

R⁹, R¹⁰, and R¹¹ can each be independently selected from group consisting of H, —C₁₋₆ haloalkyl, F, and Cl. R⁹ and R¹¹ can be H and R¹⁰ can be H, —C₁₋₆ haloalkyl, F, or Cl. R⁹ and R¹¹ can be H and R¹⁰ can be H, —CF₃, F, or Cl. R⁹ and R¹¹ can be H and R¹⁰ can be —CF₃. R⁹ and R¹¹ can be H and R¹⁰ can be F. R⁹ and R¹⁰ can be H and R¹⁰ can be Cl. R⁹, R^{1β}, and R^{1′} can be H.

FL can be attached to L via a nitrogen atom (e.g., of L). FL can be attached to L via a triazolyl or an amide (e.g., of L).

FL can have a structure selected from the group consisting of:

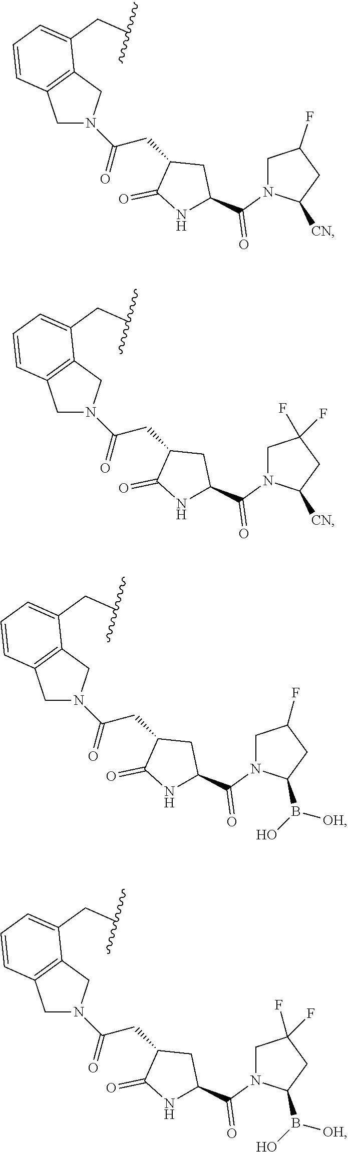 [SMILES: *Cc1cccc2c1CN(C(=O)C[C@@H]1C[C@@H](C(=O)N3CC(F)C[C@H]3C#N)NC1=O)C2 ; *Cc1cccc2c1CN(C(=O)C[C@@H]1C[C@@H](C(=O)N3CC(F)(F)C[C@H]3C#N)NC1=O)C2 ; *Cc1cccc2c1CN(C(=O)C[C@@H]1C[C@@H](C(=O)N3CC(F)C[C@H]3B(O)O)NC1=O)C2 ; *Cc1cccc2c1CN(C(=O)C[C@@H]1C[C@@H](C(=O)N3CC(F)(F)C[C@H]3B(O)O)NC1=O)C2]

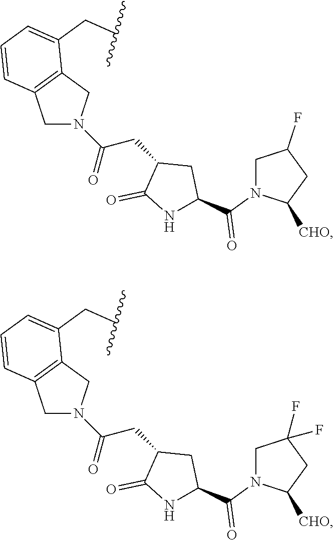 [SMILES: *Cc1cccc2c1CN(C(=O)C[C@@H]1C[C@@H](C(=O)N3CC(F)C[C@H]3C=O)NC1=O)C2 ; *Cc1cccc2c1CN(C(=O)C[C@@H]1C[C@@H](C(=O)N3CC(F)(F)C[C@H]3C=O)NC1=O)C2]

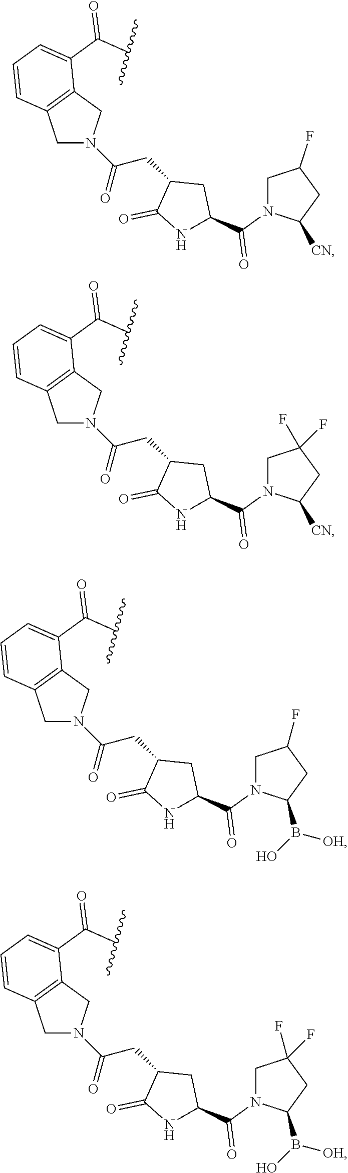 [SMILES: *C(=O)c1cccc2c1CN(C(=O)C[C@@H]1C[C@@H](C(=O)N3CC(F)C[C@H]3C#N)NC1=O)C2 ; *C(=O)c1cccc2c1CN(C(=O)C[C@@H]1C[C@@H](C(=O)N3CC(F)(F)C[C@H]3C#N)NC1=O)C2 ; *C(=O)c1cccc2c1CN(C(=O)C[C@@H]1C[C@@H](C(=O)N3CC(F)C[C@H]3B(O)O)NC1=O)C2 ; *C(=O)c1cccc2c1CN(C(=O)C[C@@H]1C[C@@H](C(=O)N3CC(F)(F)C[C@H]3B(O)O)NC1=O)C2]

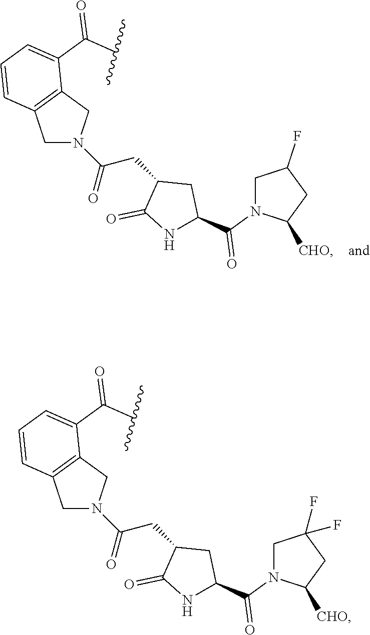 [SMILES: *C(=O)c1cccc2c1CN(C(=O)C[C@@H]1C[C@@H](C(=O)N3CC(F)C[C@H]3C=O)NC1=O)C2 ; "and" ; *C(=O)c1cccc2c1CN(C(=O)C[C@@H]1C[C@@H](C(=O)N3CC(F)(F)C[C@H]3C=O)NC1=O)C2]

Alternatively, FL can have a structure selected from the group consisting of:

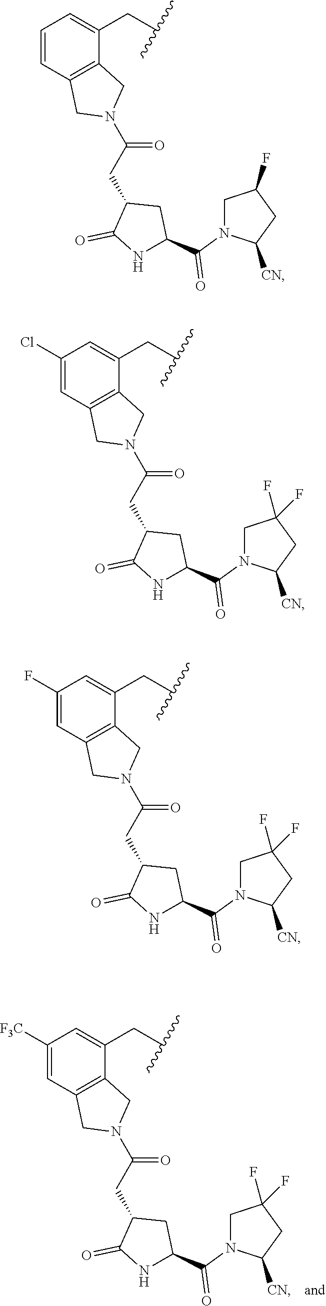 [SMILES: *Cc1cccc2c1CN(C(=O)C[C@@H]1C[C@@H](C(=O)N3C[C@@H](F)C[C@H]3C#N)NC1=O)C2 ; *Cc1cc(Cl)cc2c1CN(C(=O)C[C@@H]1C[C@@H](C(=O)N3CC(F)(F)C[C@H]3C#N)NC1=O)C2 ; *Cc1cc(F)cc2c1CN(C(=O)C[C@@H]1C[C@@H](C(=O)N3CC(F)(F)C[C@H]3C#N)NC1=O)C2 ; *Cc1cc(C(F)(F)F)cc2c1CN(C(=O)C[C@@H]1C[C@@H](C(=O)N3CC(F)(F)C[C@H]3C#N)NC1=O)C2]

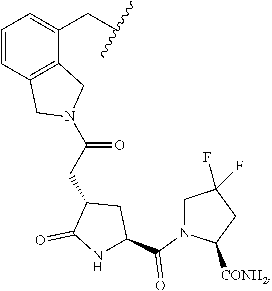 [SMILES: *Cc1cccc2c1CN(C(=O)C[C@@H]1C[C@@H](C(=O)N3CC(F)(F)C[C@H]3C(N)=O)NC1=O)C2]

FL can have a binding affinity to a FAP (e.g., FAPα) in the range of about 1 nM to about 25 nM, such as 1 nM to about 25 nM or about 1 nM to 25 nM.

L is a linker, which binds an FL to IA. L can be any suitable linker and forms a chemical bond with FL and/or IA. L can comprise atoms selected from C, N, O, S, Si, and P; C, N, O, S, and P; or C, N, O, and S. L can comprise one or more linker groups, such as, for example, in the range from about 2 to about 100 atoms in a contiguous backbone. L can comprise one or more linker groups independently selected from the group consisting of alkyl(ene), heteroalkyl(ene), heterocycloalkyl(ene), heteroaryl, aryl, alkoxy, thioether, disulfide, carboxylic acid, anhydride, carbonate, carbamate, thioether, sugar, and peptide. L can comprise one or more linker groups independently selected from the group consisting of polyethylene glycol (PEG), alkyl(ene), amide, carboxylic acid, anhydride, carbonate, ester, carbamate, thioether, phenyl, sugar, peptide, and triazole. L can comprise one or more linker groups, each linker group independently selected from the group consisting of PEG, alkyl(ene), disulfide, amide, carboxylic acid, ester, and carbonate. L can comprise one or more linker groups, each linker group independently selected from the group consisting of PEG, alkyl(ene), disulfide, and amide. L can comprise one or more linker groups, each linker group independently selected from the group consisting of alkyl(ene), disulfide, and amide. L can comprise one or more linker groups, each linker group independently selected from the group consisting of amide, alkyl(ene), PEG, phenyl, and triazole. L can comprise one or more linker groups, each linker group independently selected from the group consisting of PEG, alkyl(ene), and amide. L can comprise one or more linker groups, each linker group independently selected from the group consisting of alkyl(ene) and amide. L can comprise one or more linker groups, each linker group independently selected from the group consisting of PEG and amide. L can comprise one or more triazole linker groups. L can comprise one or more disulfide linker groups. L can comprise one or more amide linker groups. L can comprise one or more PEG linker groups.

L can be releasable or non-releasable. Non-releasable can be preferred. L can bind more than one FL, such as one, two or three FLs. While L can bind more than one imaging agent, L binding of one IA can be preferred.

The IA can be (CM), in which M is a metal (e.g., gadolinium (Gd³⁺), manganese (Mn²⁺), or dysprosium. (Dy³⁺) and C is a chelator. C can be selected from the group consisting of DOTA (1,4,7,10-tetraazacyclododecane-1,4,7,14-tetraacetic acid), TETA (1,4,8,11-tetraazacyclotetradecane-1,4,8,11-tetraacetic acid), SarAr (1-N-(4-Aminobenzyl)-3,6,10,13,16,19-hexaazabicyclo[6.6.6]-eicosane-1,8-diamine), NOTA (1,4,7-triazacyclononane-1,4,7-triacetic acid), NETA (4-[2-(bis-carboxymethylamino)-ethyl]-7-carboxymethyl-[1,4,7]triazonan-1-yl) acetic acid, DTPA (diethylenetriaminepentaacetic acid), or a derivative of any of the foregoing.

C can have a structure selected from:

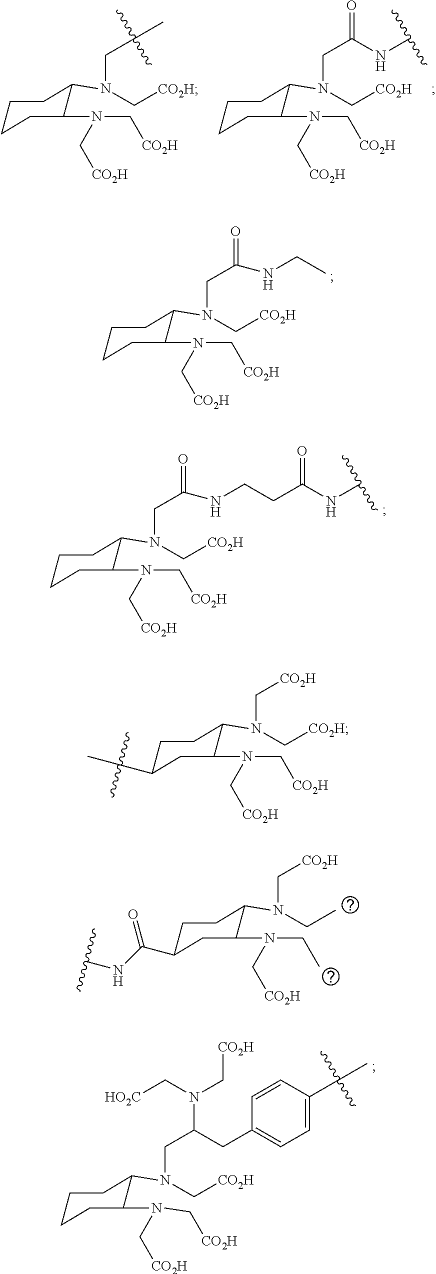

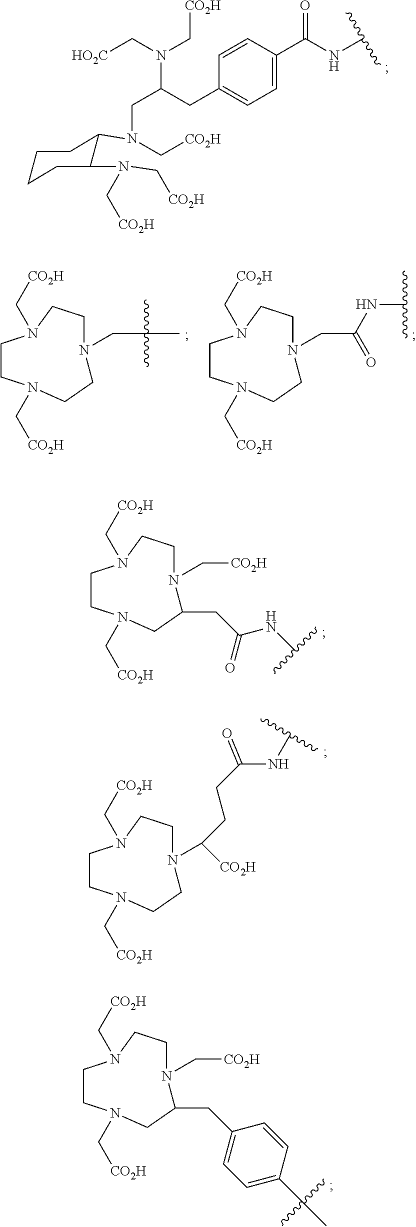 [SMILES: *NC(=O)c1ccc(CC(CN(CC(=O)O)C2CCCCC2N(CC(=O)O)CC(=O)O)N(CC(=O)O)CC(=O)O)cc1 ; *CN1CCN(CC(=O)O)CCN(CC(=O)O)CC1 ; *NC(=O)CN1CCN(CC(=O)O)CCN(CC(=O)O)CC1 ; *NC(=O)CC1CN(CC(=O)O)CCN(CC(=O)O)CCN1CC(=O)O ; *NC(=O)CCC(C(=O)O)N1CCN(CC(=O)O)CCN(CC(=O)O)CC1 ; *c1ccc(CC2CN(CC(=O)O)CCN(CC(=O)O)CCN2CC(=O)O)cc1]

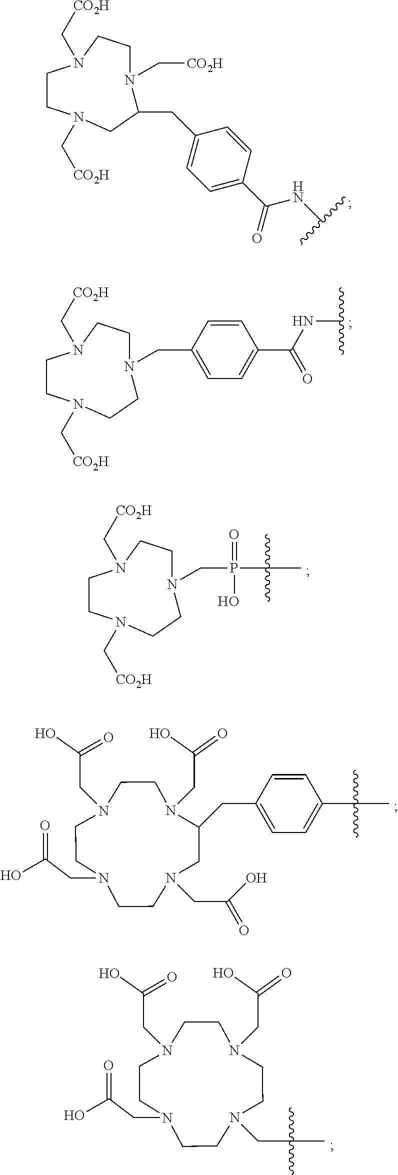

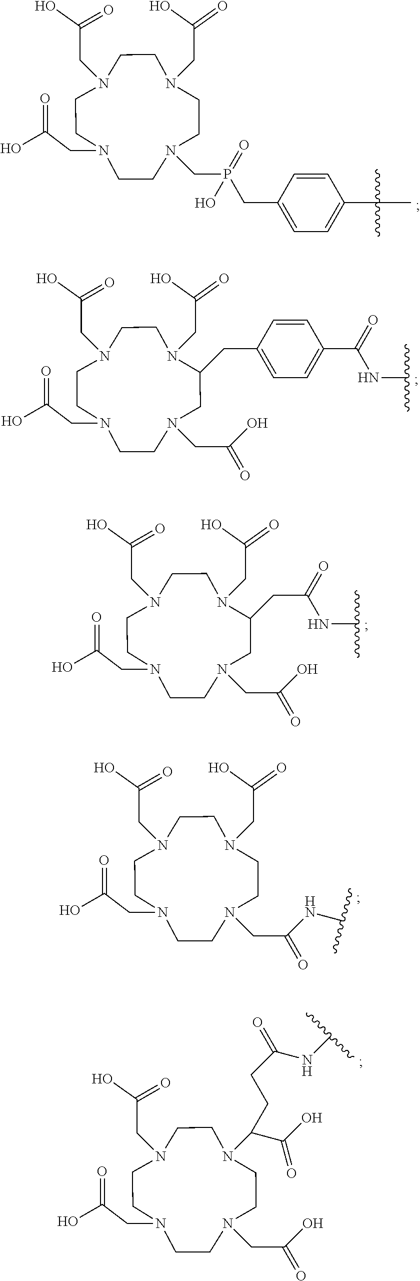 [SMILES: *c1ccc(CP(=O)(O)CN2CCN(CC(=O)O)CCN(CC(=O)O)CCN(CC(=O)O)CC2)cc1 ; *NC(=O)c1ccc(CC2CN(CC(=O)O)CCN(CC(=O)O)CCN(CC(=O)O)CCN2CC(=O)O)cc1 ; *NC(=O)CC1CN(CC(=O)O)CCN(CC(=O)O)CCN(CC(=O)O)CCN1CC(=O)O ; *NC(=O)CN1CCN(CC(=O)O)CCN(CC(=O)O)CCN(CC(=O)O)CC1 ; *NC(=O)CCC(C(=O)O)N1CCN(CC(=O)O)CCN(CC(=O)O)CCN(CC(=O)O)CC1]

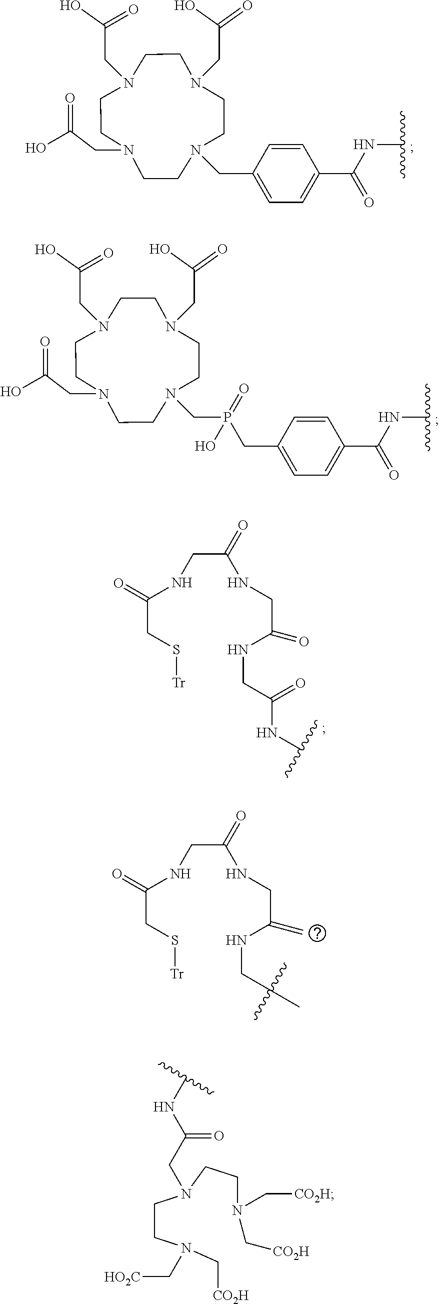

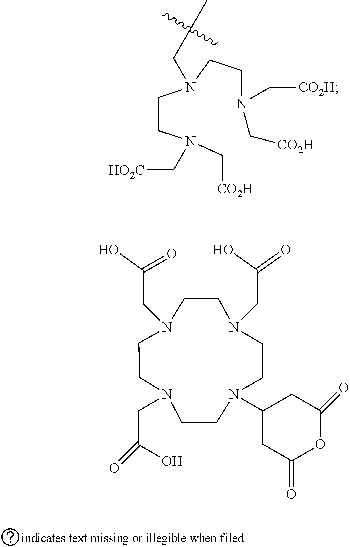 [SMILES: *CN(CCN(CC(=O)O)CC(=O)O)CCN(CC(=O)O)CC(=O)O ; O=C(O)CN1CCN(CC(=O)O)CCN(C2CC(=O)OC(=O)C2)CCN(CC(=O)O)CC1]

The MRI agent can be a paramagnetic contrast agent. The paramagnetic contrast agent can comprise gadolinium (Gd³⁺), manganese (Mn²⁺), or dysprosium (Dy³⁺). The paramagnetic contrast agent can be Gd-IOTA (gadoterate dotarem), Gd-HP-DO3A (gadoteridol), Gd-BT-DO3A (gadobutrol), Gd-DTPA (gadopentate dimeglumine), Gd-DTPA-BMEA (gadoversetamide), Gd-DTPA-BMA (gadodiamide), Gd-BOPTA (gadobenate dimeglumine), Gd-EOB-DTPA (gadoxetate), or Ms-325 (gadofosveset).

IA can be attached to L via a carbon atom or a nitrogen atom (e.g., of L). IA can be attached to L via a triazolyl. IA can be attached to L via an oxo (e.g., an ester). IA can be attached to L via an amide (e.g., of L).

With regard to the above conjugates, “oxo” refers to the ═O radical.

“Alkyl” generally refers to a straight or branched hydrocarbon chain radical consisting solely of carbon and hydrogen atoms, such as having from one to fifteen carbon atoms (e.g., C₁-C₁₅ alkyl). Disclosures provided herein of an “alkyl” are intended to include independent recitations of a saturated “alkyl,” unless otherwise stated. An alkyl can comprise one to thirteen carbon atoms (e.g., C₁-C₁₃ alkyl). An alkyl can comprise one to eight carbon atoms (e.g., C₁-C₈ alkyl). An alkyl can comprise one to five carbon atoms (e.g., C₃-C₅ alkyl). An alkyl can comprise one to four carbon atoms (e.g., C₃-C₄ alkyl). An alkyl can comprise one to three carbon atoms (e.g., C₁-C₃ alkyl). An alkyl can comprise one to two carbon atoms (e.g., C₁-C₂ alkyl). An alkyl can comprise one carbon atom (e.g., C₁ alkyl). An alkyl can comprise five to fifteen carbon atoms (e.g., C₅-C₁₅ alkyl). An alkyl can comprise five to eight carbon atoms (e.g., C₅-C₈ alkyl). An alkyl can comprise two to five carbon atoms (e.g., C₂-C₅ alkyl). An alkyl can comprise three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

“Alkoxy” refers to a radical bonded through an oxygen atom of the formula —O— alkyl, where alkyl is an alkyl chain as defined above.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Mickel theory. The ring system from which aryl groups are derived include, but are not limited to, groups such as benzene, fluorene, indane, indene, tetralin and naphthalene.

“Aralkyl” or “aryl-alkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. A carbocyclyl can comprise three to ten carbon atoms. A carbocyclyl can comprise five to seven carbon atoms. The carbocyclyl is attached to the rest of the molecule by a single bond. Carbocyclyl or cycloalkyl is saturated (i.e., containing single C—C bonds only) or unsaturated (i.e., containing one or more double bonds or triple bonds). Examples of saturated cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like.

“Carbocyclylalkyl” refers to a radical of the formula —R^{c}-carbocyclyl where R^{c} is an alkylene chain as defined above.

“Halo” or “halogen” refers to bromo, chloro, fluoro or iodo substituents.

“Haloalkyl” refers to an alkyl radical, as defined above, that is substituted by one or more halogen radicals, as defined above, for example, trifluoromethyl, difluoromethyl, fluoromethyl, 2,2,2-trifluoroethyl, 1-fluoromethyl-2-fluoroethyl, and the like.

The term “heteroalkyl” refers to an alkyl group as defined above in which one or more skeletal carbon atoms of the alkyl are substituted with a heteroatom (with the appropriate number of substituents or valencies—for example, —CH₂— may be replaced with —NH— or —O—). For example, each substituted carbon atom is independently substituted with a heteroatom, such as wherein the carbon is substituted with a nitrogen, oxygen, selenium, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g. —S—, —S(═O)—, or —S(═O)₂—). A heteroalkyl is attached to the rest of the molecule at a carbon atom of the heteroalkyl. A heteroalkyl is attached to the rest of the molecule at a heteroatom of the heteroalkyl. A heteroalkyl is a C₁-C₁₈ heteroalkyl. A heteroalkyl is a C₁-C₁₂ heteroalkyl. A heteroalkyl is a C₁-C₆ heteroalkyl. A heteroalkyl is a C₁-C₄ heteroalkyl. Heteroalkyl can include alkoxy, alkoxyalkyl, alkylamino, alkylaminoalkyl, aminoalkyl, heterocycloalkyl, heterocycloalkyl, and heterocycloalkylalkyl, as defined herein.

“Heteroalkylene” refers to a divalent heteroalkyl group defined above which links one part of the molecule to another part of the molecule.

“Heterocyclyl” refers to a stable 3- to 18-membered non-aromatic ring radical that can comprise two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes aromatic, fused, and/or bridged ring systems. The heteroatoms in the heterocyclyl radical are optionally oxidized. The heterocyclyl radical is partially or fully saturated. Disclosures provided herein of an “heterocyclyl” are intended to include independent recitations of heterocyclyl comprising aromatic and non-aromatic ring structures, unless otherwise stated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, 1,3-benzodioxolyl, 1,4-benzodioxanyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, indolinyl, isoindolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“Heteroaryl” refers to a radical derived from a 3- to 18-membered aromatic ring radical that can comprise two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) it electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, benzofuranyl, benzooxazolyl, benzo[d]thiazolyl, benzothiadiazolyl, benzo[b][1,4]dioxepinyl, benzo[b][1,4]oxazinyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl).

The compounds can contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

One of ordinary skill in the art will further appreciate that the compounds can be “deuterated,” meaning one or more hydrogen atoms can be replaced with deuterium. As deuterium and hydrogen have nearly the same physical properties, deuterium substitution is the smallest structural change that can be made.

Further provided is a pharmaceutical composition. The pharmaceutical composition comprises an above-described conjugate and a pharmaceutically acceptable carrier. The pharmaceutical composition can comprise a plurality of conjugates and a pharmaceutically acceptable carrier. The composition is suitable for administration to a subject for diagnostic, mapping, and/or therapeutic applications. By “suitable” for administration is meant that administration of the conjugated nanoparticle/composition to a subject will not result in unacceptable toxicity, including allergenic responses and disease states.

By “pharmacologically acceptable” is meant being suitable for administration to a subject. In other words, administration of the relevant material to a subject should not result in unacceptable toxicity, including allergenic responses and disease states.

As a guide only, a person skilled in the art may consider “pharmacologically acceptable” as an entity approved by a regulatory agency of a federal or state government or listed in the US Pharmacopeia or other generally recognised pharmacopeia for use in animals, and more particularly humans.

Having said that, those skilled in the art will appreciate the suitability of the conjugate or composition for administration to a subject and whether it or its constituent components would be considered pharmacologically acceptable, will to some extent depend upon the mode of administration selected. Thus, the mode of administration may need to be considered when evaluating whether a composition or constituent component thereof is suitable for administration to a subject or if it is pharmacologically acceptable.

Pharmaceutical compositions can be prepared by combining one or more compounds with a pharmaceutically acceptable carrier and, optionally, one or more additional pharmaceutically active agents.

Compositions can comprise one or more pharmacologically acceptable additives known to those in the art. For example, the liquid carrier may comprise one or more additives such as wetting agents, de-foaming agents, surfactants, buffers, electrolytes, preservatives, colourings, flavourings, and sweeteners.

The particular nature of a liquid carrier and any additive (if present) will in part depend upon the intended application of the composition. A suitable liquid carrier and additive (if present) can be selected for the intended application of the composition.

Where the conjugate or compositions are suitable for parenteral administration, they will generally be in the form of an aqueous or non-aqueous isotonic sterile injection solution that may contain one or more of an anti-oxidant, buffer, bactericide or solute which renders the composition isotonic with the blood of the intended subject. Such compositions can be presented in unit-dose or multi-dose sealed containers, for example, ampoules and vials.

Still further provided is a method of imaging cells, a tissue, or an organ, any of which express(es) FAP, in a subject. By the term “subject” is meant either an animal or human subject. By “animal” is meant primates, livestock animals (including cows, horses, sheep, pigs and goats), companion animals (including dogs, cats, rabbits and guinea pigs), and captive wild animals (including those commonly found in a zoo environment). Laboratory animals such as rabbits, mice, rats, guinea pigs and hamsters are also contemplated as they may provide a convenient test system. Given that FAP homologs have been found in zebrafish and amphibians, i.e., two species of the Xenopus genus, the subject, in certain instances, could be a fish or an amphibian.

The subject can be a human or a mammal of economic importance and/or social importance to humans, for instance, carnivores other than humans (e.g., cats and dogs), swine (e.g., pigs, hogs, and wild boars), ruminants (e.g., cattle, oxen, sheep, giraffes, deer, goats, bison, and camels), horses, and birds including those kinds of birds that are endangered and kept in zoos, and fowl, more particularly domesticated fowl, e.g., poultry, such as turkeys, chickens, ducks, geese, guinea fowl, and the like, as they are also of economic importance to humans.

The term “subject” does not denote a particular age. Thus, adult, juvenile and newborn subjects are covered.

The terms “subject,” “individual” and “patient” may be used interchangeably herein.

In certain embodiments, the subject is a mammal. In certain embodiments, the subject is a human.

The method comprises administering to the subject an above-described conjugate or a pharmaceutical composition comprising the conjugate and a pharmaceutically acceptable carrier, and having the cells, the tissue, or the organ in the subject imaged with magnetic resonance imaging.

Examples of suitable therapeutic or diagnostic applications include magnetic resonance imaging (MRI), MRI guided external beam radiotherapy, MRI guided focal ablation, MRI/Ultrasound fusion focal ablation, MRI guided biopsy, MRI/Ultrasound fusion guided biopsy, MRI guided surgery, MRI guided brachytherapy, and MRI guided infrared camera guided biopsy or therapy.

The conjugate or composition can be used in conjunction with other in vivo imaging techniques including, but not limited to, ultrasound, X-ray, optical imaging, Computed Tomography (CT), Single Photon Emission Computed Tomography (SPECT), Positron Emission Tomography (PET) and Fluorescence Resonance Energy Transfer (FRET).

The subject can have cancer, and the cells, the tissue, or the organ with cancer can be imaged.

The subject can have a tumor with a microenvironment, and the method can further comprise obtaining a map of the microenvironment of the tumor, such as when the conjugate accumulates in the tumor microenvironment, e.g., along the margin of the tumor, such as stromal cells. The tumor can be mapped in situ before and/or after tumor resection.

The composition comprising the conjugate allows for detection of cells expressing FAP, such as cells within the tumor microenvironment (e.g., tumor-associated stromal cells and cancer cells) associated with solid tumors, such as prostate cancer, glioblastoma, pancreatic cancer, colorectal cancer, breast cancer and lung cancer. By specifically binding to FAP expressed by the cells of the tumor microenvironment, the conjugate is useful for the identification of the boundaries and margins of tissue that is affected by cancer (i.e., tumor mapping). It is also contemplated herein that the conjugate and compositions can be useful for the detection (i.e., diagnosis) of cancer or as part of the treatment of cancer. For example, the conjugate can be used for tumor mapping prior to the commencement of treatment, such as focal therapy, radiotherapy, proton therapy or brachytherapy. Furthermore, by accurately mapping the tumor, including regions of the tumor microenvironment, surgical resection of the tumor can be performed with more accuracy to limit undesirable side effects and minimising the risk of suboptimal debulking of the tumor mass.

As used herein, the expression “tumor microenvironment” refers to a heterogeneous population of non-cancerous cells surrounding and/or infiltrating a tumor, which are essential to the functionality, physiology and metastasis of the tumor. The skilled person will appreciate that the tumor microenvironment comprises a range of different cell types that may differ based on the size, location, type and stage of a tumor, illustrative examples of which include fibroblasts, pericytes, adipocytes, mesenchymal stromal cells (MSCs), cancer cells and endothelial cells, and combinations thereof (such as pericytes and endothelial cells). While the cells of the tumor microenvironment are non-cancerous, tumors recruit and or regulate such cells to provide a favourable environment to facilitate cancer growth. Accordingly, cells comprised within the tumor microenvironment may be referred to as “cancer-associated” or “tumor-associated”.

The subject can have a tumor, and the method can further comprise obtaining a measurement of a gross target volume and/or a clinical target volume for treatment, such as prior to treatment.

When the conjugates and pharmaceutical compositions comprising the conjugates are administered for imaging, i.e., magnetic resonance imaging, the conjugates and compositions may be administered by any suitable route including, for example, intravenously, intraperitoneally, subcutaneously, intracranially, intradermally, intramuscularly, intraocularly, intrathecally, intracerebrally, and intranasally. Typically, the conjugates and compositions are administered intravenously or orally. The conjugates and compositions are administered orally for gastrointestinal scans.

The amount of conjugate or composition administered should be the smallest amount sufficient to generate a clinically useful image. Amounts of currently available contrast agents can be used as a guide in determining the amounts of the conjugates and compositions to be used.

The method can further comprise treating the subject, or having the subject treated, for cancer. For example, the method can further comprise administering an effective amount of a treatment for cancer at a site where the conjugate accumulates. The treatment can be any suitable treatment, such as surgery, radiotherapy, brachytherapy, photodynamic therapy, photothermal therapy, focal ablation therapy including cryoablation, focal laser ablation and high-frequency ultrasound ablation, chemotherapy, and immunotherapy.

The therapeutic regimen for the treatment of cancer can be determined by a person skilled in the art and will typically depend on factors including, but not limited to, the type, size, stage and receptor status of the tumor in addition to the age, weight and general health of the subject. Another determinative factor can be the risk of developing recurrent disease. For instance, for a subject identified as being at high risk or higher risk or developing recurrent disease, a more aggressive therapeutic regimen can be prescribed as compared to a subject who is deemed at a low or lower risk of developing recurrent disease. Similarly, for a subject identified as having a more advanced stage of cancer, for example, stage III or IV disease, a more aggressive therapeutic regimen can be prescribed as compared to a subject that has a less advanced stage of cancer.

The terms “treat,” “treatment,” and “treating” refer to any and all uses which remedy a condition or symptom, or otherwise prevent, hinder, retard, abrogate or reverse the onset or progression of cancer or other undesirable symptoms in any way whatsoever. Thus, the term “treating,” and the like, is to be considered in its broadest possible context. For example, treatment does not necessarily imply that a subject is treated until total recovery or cure. In conditions that display or are characterized by multiple signs or symptoms, the treatment need not necessarily remedy, prevent, hinder, retard, abrogate or reverse all signs or symptoms, but can remedy, prevent, hinder, retard, abrogate or reverse one or more signs or symptoms.

The expression “therapeutically effective amount” means the amount of conjugate when administered to a mammal, in particular a human, in need of such treatment, is sufficient to treat cancer. The precise amount of conjugate to be administered can be determined by a physician with consideration of individual differences in age, weight, tumor size, extent of infection or metastasis, and condition of the subject.

By “administration” of the conjugate or composition to a subject is meant that the conjugate or composition is presented such that the conjugate can be transferred to the subject. There is no particular limitation on the mode of administration, but this will generally be by way of oral, parenteral (including subcutaneous, intradermal, intramuscular, intravenous, intracerebrally, intranasally, intrathecal, and intraspinal), inhalation (including nebulisation), topical, rectal and vaginal modes. The conjugate or composition may also be administered directly into a tumor and/or into tissue adjacent one or more segments of a tumor or administered directly into blood vessels.

The conjugate can be administered in, as appropriate, a treatment or diagnostic effective amount. A treatment or diagnostic effective amount is intended to include an amount which, when administered according to the desired dosing regimen, achieves a desired therapeutic or diagnostic effect, including one or more of: alleviating the symptoms of, preventing or delaying the onset of, inhibiting or slowing the progression of, diagnosing, or halting or reversing altogether the onset or progression of a particular condition being treated and/or assessed.

Suitable dosage amounts and dosing regimens to achieve this can be determined by the attending physician and can depend on the particular condition being treated or diagnosed, the severity of the condition as well the general age, health and weight of the subject.

Dosing can occur at intervals of minutes, hours, days, weeks, months or years or continuously over any one of these periods. Suitable dosages of the particulate material per se can lie within the range of about 0.1 ng per kg of body weight to 1 g per kg of body weight per dosage. The dosage can be in the range of 1 μg to 1 g per kg of body weight per dosage, such as is in the range of 1 mg to 1 g per kg of body weight per dosage. In one embodiment, the dosage can be in the range of 1 mg to 500 mg per kg of body weight per dosage. In another embodiment, the dosage can be in the range of 1 mg to 250 mg per kg of body weight per dosage. In yet another embodiment, the dosage can be in the range of 1

The mg to 100 mg per kg of body weight per dosage, such as up to 50 mg per body weight per dosage.

Conjugate or compositions can be administered in a single dose or a series of doses.

### EXAMPLES

The following examples serve to illustrate the present disclosure and are not intended to limit the scope of the claimed invention in any way.

Additional examples of compounds of the disclosure include compounds of the formula:

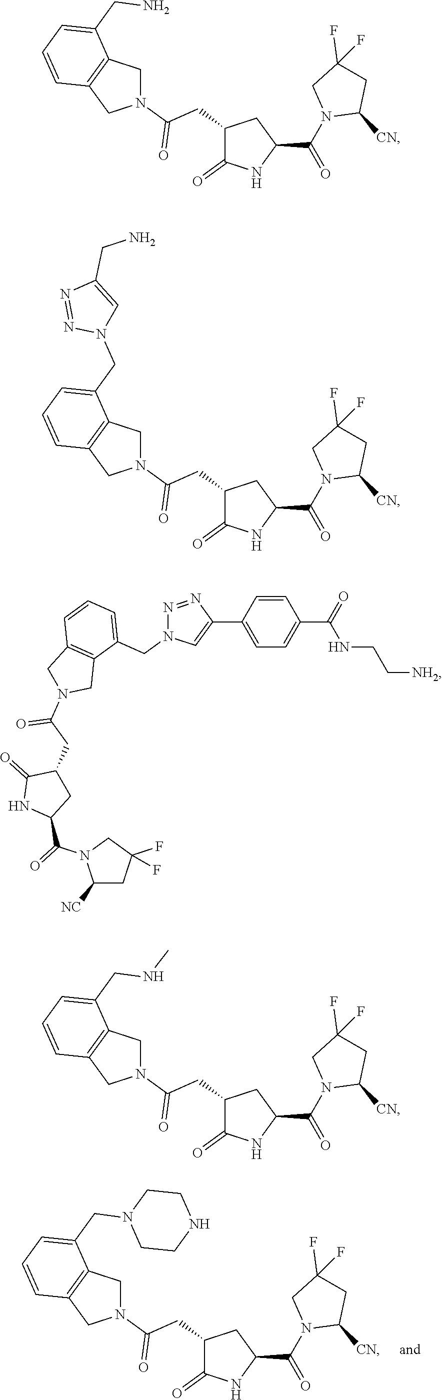

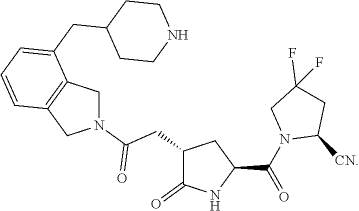 [SMILES: N#C[C@@H]1CC(F)(F)CN1C(=O)[C@@H]1C[C@@H](CC(=O)N2Cc3cccc(CC4CCNCC4)c3C2)C(=O)N1]

Such compounds fall under the general formula (II) and can be used to form conjugates of the formula (III):

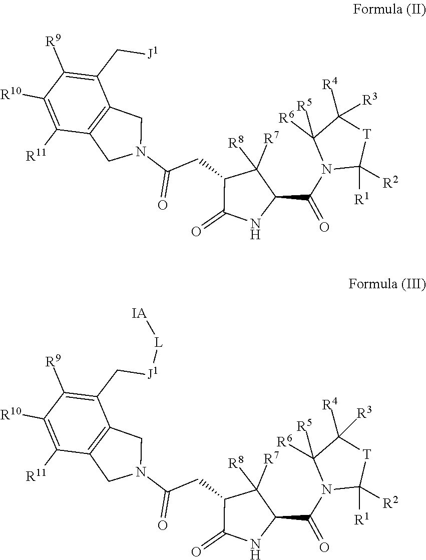

wherein J¹ comprises or is an amino group such as —NHR; wherein R is H or alkyl; comprises or is a heterocyclyl group, such as a piperidinyl or a piperazinyl group; or comprises or is J²-J³-J⁴, wherein J² is heterocyclyl; J³ is absent arylamido or amidoaryl; and J⁴ is aminoalkyl (e.g., -alkyl-NHR).

Additional examples of compounds of the disclosure also include compounds of the formula:

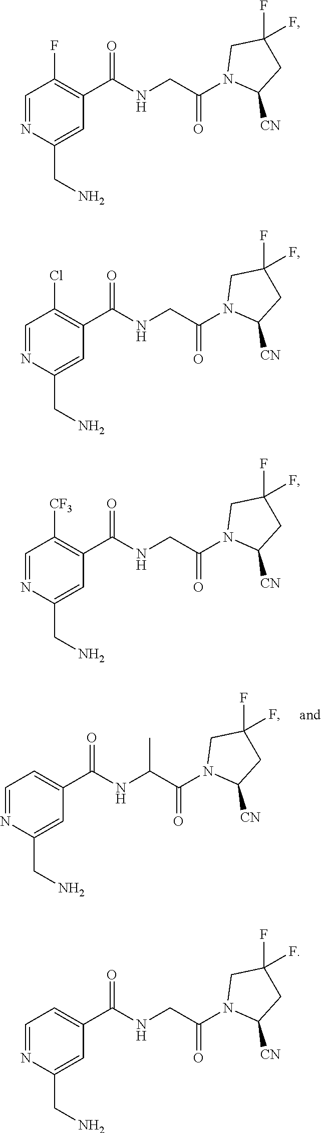 [SMILES: N#C[C@@H]1CC(F)(F)CN1C(=O)CNC(=O)c1cc(CN)ncc1F ; N#C[C@@H]1CC(F)(F)CN1C(=O)CNC(=O)c1cc(CN)ncc1Cl ; N#C[C@@H]1CC(F)(F)CN1C(=O)CNC(=O)c1cc(CN)ncc1C(F)(F)F ; CC(NC(=O)c1ccnc(CN)c1)C(=O)N1CC(F)(F)C[C@H]1C#N ; N#C[C@@H]1CC(F)(F)CN1C(=O)CNC(=O)c1ccnc(CN)c1]

Such compounds fall under the general formula (IV) and can be used to form conjugates of the formula (V):

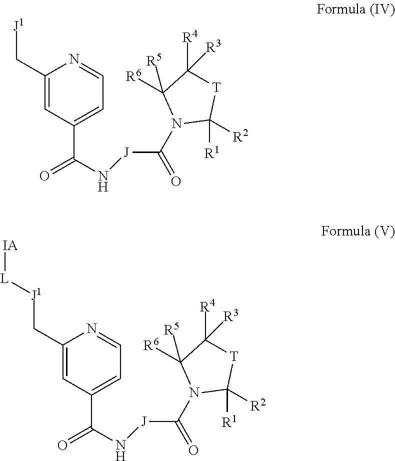

Additional examples of compounds of the disclosure also include compounds of the formula:

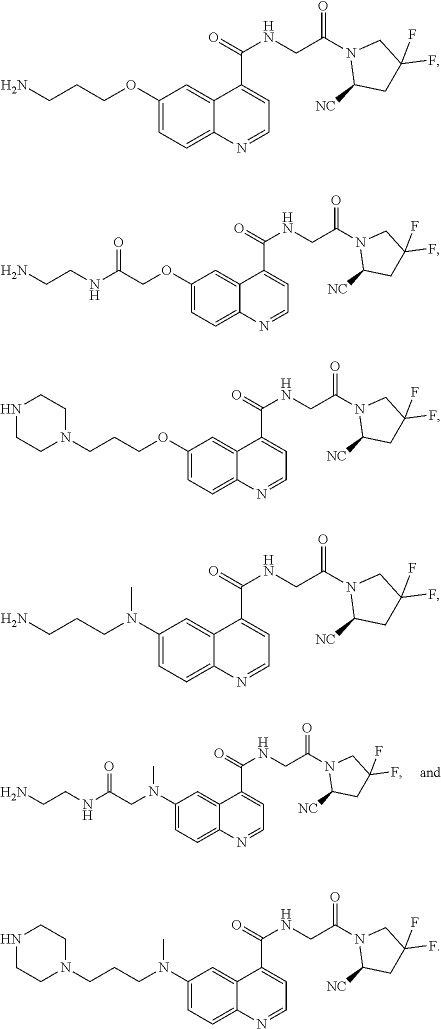 [SMILES: N#C[C@@H]1CC(F)(F)CN1C(=O)CNC(=O)c1ccnc2ccc(OCCCN)cc12 ; N#C[C@@H]1CC(F)(F)CN1C(=O)CNC(=O)c1ccnc2ccc(OCC(=O)NCCN)cc12 ; N#C[C@@H]1CC(F)(F)CN1C(=O)CNC(=O)c1ccnc2ccc(OCCCN3CCNCC3)cc12 ; CN(CCCN)c1ccc2nccc(C(=O)NCC(=O)N3CC(F)(F)C[C@H]3C#N)c2c1 ; CN(CC(=O)NCCN)c1ccc2nccc(C(=O)NCC(=O)N3CC(F)(F)C[C@H]3C#N)c2c1 ; CN(CCCN1CCNCC1)c1ccc2nccc(C(=O)NCC(=O)N3CC(F)(F)C[C@H]3C#N)c2c1]

Such compounds fall under the general formula (IV) and can be used to form conjugates of the formula (V):

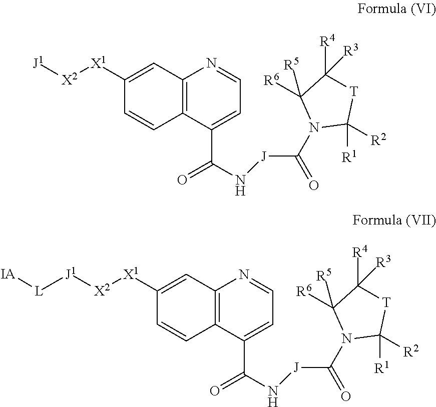

wherein:
X¹ is O or NR; X² is alkyl or alkylamidoalkyl.

### Example 1

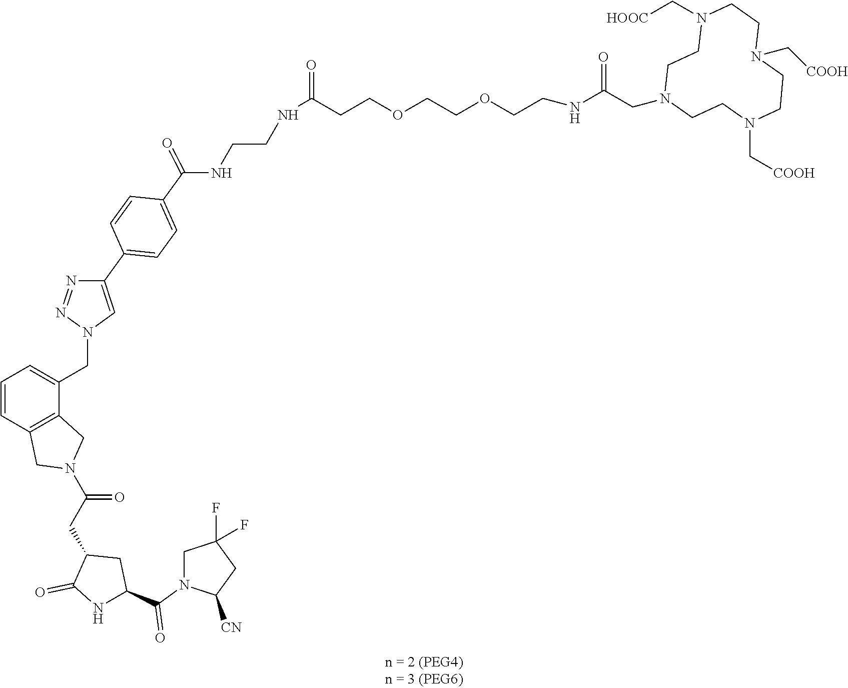 [SMILES: N#C[C@@H]1CC(F)(F)CN1C(=O)[C@@H]1C[C@@H](CC(=O)N2Cc3cccc(Cn4cc(-c5ccc(C(=O)NCCNC(=O)CCOCCOCCNC(=O)CN6CCN(CC(=O)O)CCN(CC(=O)O)CCN(CC(=O)O)CC6)cc5)nn4)c3C2)C(=O)N1]

### Example 2

### Binding to Fibroblast Activation Protein (FAP)

The binding affinity of conjugates (e.g., with a metal M) to FAP is evaluated in vitro by comparing their cellular uptake in a human primary glioblastoma cell line U87. U87 cells are cultured in T75 cell culture flasks in RPMI medium supplemented with 10% Fetal Bovine Serum (FBS) and 1% Penicillin-Streptomycin and are kept in a 37° C., 5% CO₂ incubator. Cells are passaged when 70-80% confluence is reached. To measure the cellular uptake and test the binding affinity of the conjugates, the U87 cells are seeded in a 6-well plate at 2.5×10⁵ cell density in cell culture medium supplemented as described above. Plates are placed in a 37° C., 5% CO₂ incubator and allowed to adhere for 24h. Transforming growth factor beta (TGF-13) at concentration 20 ng·mL⁻¹ is added to the adherent cell monolayer, and cells are kept for 48 h in a 37° C., 5% CO₂ incubator. TGF-β stimulates the expression of FAP in U87 cells and allows for them to be used as a tool to evaluate the binding affinity of the conjugates.

All conjugates are prepared at a concentration of 0.150 mg Fe·mL⁻¹ in cell culture media suspensions. Conjugate suspension (1 mL) is added to each well after removing the TGF-β stimulation media. U87 cells are incubated with the conjugate suspensions for 24 h in a 37° C., 5% CO₂ incubator. Cells are washed 3 times with PBS and detached using trypsin. Cell pellets are collected via centrifugation at 500 g for 5 min. Cell pellets are washed two more times with PBS and finally dried at 60° C. overnight using a heating block. Dried cell pellets are digested with trace metal grade nitric and hydrochloric acid (1:1 ratio by volume), and samples are diluted with water to a total volume of 10 mL. Iron concentration is measured with inductively coupled plasma optical emission spectroscopy (ICP-OES). The acquired data are analyzed and tested for significance with one-way ANOVA.

### Example 3

### Animal Testing of Conjugates for the Delineation of Prostate Tumors

Male NOD SCID gamma mice aged 6-8 weeks are injected with human prostate cancer cells (LNCaP) directly into the prostate. After 4-6 weeks of tumor growth, the mice are injected with 40 mg/kg of 15 mg/ml of magnetic nanoparticles with FAP5 targeting moiety or no targeting moiety (control) into the tail vein.

After a 24-hour uptake period, mice are anaesthetized with 2% isoflurane in oxygen and are intraperitoneally injected with an overdose of pentobarbital prior to transcardial perfusion with 4% paraformaldehyde. Mice are postfixed in 4% paraformaldehyde prior to storage in PBS with 0.1% sodium azide. Mice undergo whole body ex vivo 12-weighted multiple graded echo sequence acquired using a 16.4T Bruker Avance scanner.

Resected prostate tumors previously fixed in 10% neutral-buffered formalin are mounted in paraffin blocks. Sections (5 μm) are cut and stained with Prussian Blue to visualize the presence of iron nanoparticles.

### Definitions

Unless defined otherwise, all technical and scientific terms used herein have the same meaning as commonly understood by one of skill in the chemical and biological arts. Additionally, as used in this specification and the appended claims, the singular forms “a”, “an” and “the” include plural referents unless the content clearly dictates otherwise. Thus, for example, where a compound/composition is substituted with “an” alkyl or aryl, the compound/composition is optionally substituted with at least one alkyl and/or at least one aryl. Furthermore, unless specifically stated otherwise, the term “about” refers to a range of values plus or minus 10% for percentages and plus or minus 1.0 unit for unit values, for example, about 1.0 refers to a range of values from 0.9 to 1.1.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, where a compound/composition is substituted with “an” alkyl or aryl, the compound/composition is optionally substituted with at least one alkyl and/or at least one aryl. Furthermore, unless specifically stated otherwise, the term “about” refers to a range of values plus or minus 10% for percentages and plus or minus 1.0 unit for unit values, for example, about 1.0 refers to a range of values from 0.9 to 1.1.

The terms and expressions, which have been employed, are used as terms of description and not of limitation. Where certain terms are defined and are otherwise described or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings may be used in the “Detailed Description,” such use is solely for ease of reference and is not intended to limit any disclosure made in one section to that section only; rather, any disclosure made under one subheading is intended to constitute a disclosure under each and every other subheading.

It is recognized that various modifications are possible within the scope of the claimed invention. Thus, although the present invention has been specifically disclosed in the context of preferred embodiments and optional features, those skilled in the art may resort to modifications and variations of the concepts disclosed herein. Such modifications and variations are considered within the scope of the invention as claimed herein.

What is claimed is:

1. A conjugate comprising:
FL-L-IA,
wherein FL is a radical of a small molecule ligand that specifically binds with fibroblast activation protein (FAP),
L is a linker, which binds an FL to IA, and
IA is a radical of a magnetic resonance imaging (MRI) agent,
or a pharmaceutically acceptable salt thereof.

2. The conjugate of claim 1, or a pharmaceutically acceptable salt thereof, wherein FL has a structure of formula I: 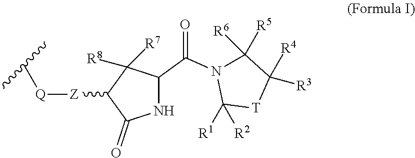
wherein Q is aryl, heteroaryl, or heterocyclyl;
Z is a bond, substituted or unsubstituted C₁-C₃ alkylene, substituted or unsubstituted heteroalkylene, amino, —O—, or —S—;
T is substituted or unsubstituted methylene, substituted or unsubstituted amino, —O—, or —S—;
R¹ and R² are each independently selected from the group consisting of —H, —CN, CHO, —B(OH)₂, —C(O)alkyl, —C(O)aryl-, —OC—C(O)aryl, —C═C—S(O)₂ aryl, —CO2H, —SO₃H, SO₂NH₂, —PO₃H₂, —SO₂F, —CONH₂, and 5-tetrazolyl;
R³ and R⁴ are each independently selected from the group consisting of —H, —OH, F, Q, Br, I, —C₁₋₆ alkyl, —O—C₁₋₆ alkyl, and —S—C₁₋₆ alkyl; and
R⁵, R⁶, R⁷, and R⁸ are each independently selected from the group consisting of H,
alkyl, and halo.

3. The conjugate of claim 1, or a pharmaceutically acceptable salt thereof, wherein FL has a structure of formula II: 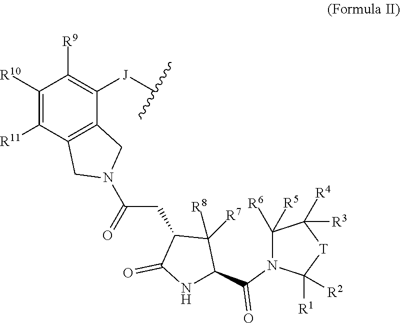
wherein:
T is substituted or unsubstituted methylene, substituted or unsubstituted amino, —O—, or —S—;
J is C(R^{J})₂, wherein each R^{J} is independently H or alkyl, or both R^{J}, when taken together, form oxo;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, SO₂NH₂, —PO₃H₂, —SO₂F, —CONH₂, and 5-tetrazolyl;
R³ and R⁴ are each independently selected from the group consisting of H, —OH, F, Q, Br, I, —C₁₋₆, alkyl, —O—C₁₋₆ alkyl, and —S—C₁₋₆ alkyl, wherein Q is aryl, heteroaryl, or heterocyclyl;
R⁵, R⁶, R⁷, and R⁸ are each independently selected from group consisting of H, alkyl, and halo; and
R⁹, R¹⁰, and R¹¹ are each independently selected from group consisting of H, C₁₋₆ alkyl, —C₁₋₆ haloalkyl, —O—C₁₋₆ alkyl, —S—C₁₋₆ alkyl, F, Q, Cl, Br and I.

4. The conjugate of claim 1, or a pharmaceutically acceptable salt thereof, wherein FL has a structure selected from the group consisting of

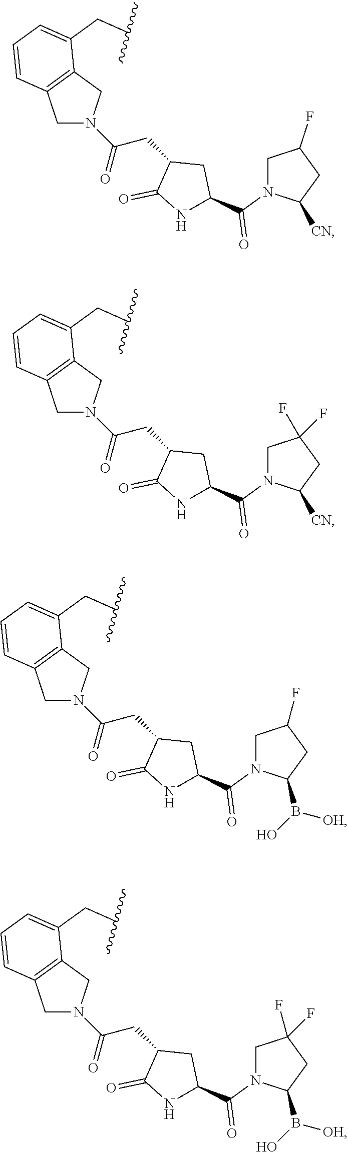 [SMILES: *Cc1cccc2c1CN(C(=O)C[C@@H]1C[C@@H](C(=O)N3CC(F)C[C@H]3C#N)NC1=O)C2 ; *Cc1cccc2c1CN(C(=O)C[C@@H]1C[C@@H](C(=O)N3CC(F)(F)C[C@H]3C#N)NC1=O)C2 ; *Cc1cccc2c1CN(C(=O)C[C@@H]1C[C@@H](C(=O)N3CC(F)C[C@H]3B(O)O)NC1=O)C2 ; *Cc1cccc2c1CN(C(=O)C[C@@H]1C[C@@H](C(=O)N3CC(F)(F)C[C@H]3B(O)O)NC1=O)C2]

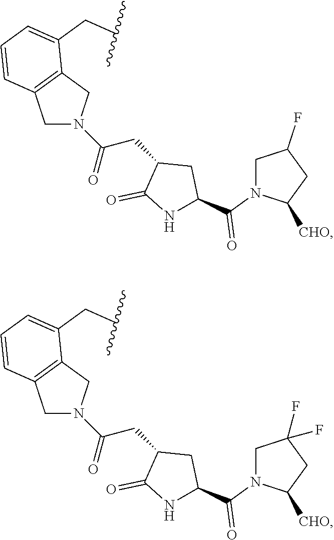 [SMILES: *Cc1cccc2c1CN(C(=O)C[C@@H]1C[C@@H](C(=O)N3CC(F)C[C@H]3C=O)NC1=O)C2 ; *Cc1cccc2c1CN(C(=O)C[C@@H]1C[C@@H](C(=O)N3CC(F)(F)C[C@H]3C=O)NC1=O)C2]

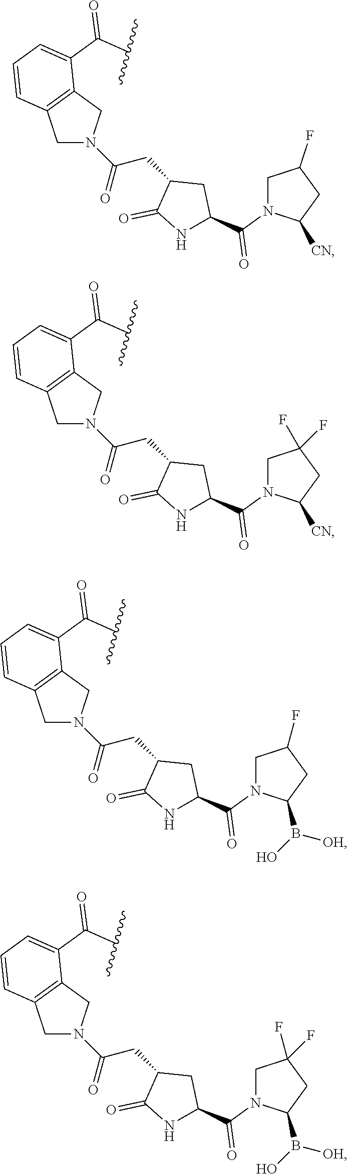 [SMILES: *C(=O)c1cccc2c1CN(C(=O)C[C@@H]1C[C@@H](C(=O)N3CC(F)C[C@H]3C#N)NC1=O)C2 ; *C(=O)c1cccc2c1CN(C(=O)C[C@@H]1C[C@@H](C(=O)N3CC(F)(F)C[C@H]3C#N)NC1=O)C2 ; *C(=O)c1cccc2c1CN(C(=O)C[C@@H]1C[C@@H](C(=O)N3CC(F)C[C@H]3B(O)O)NC1=O)C2 ; *C(=O)c1cccc2c1CN(C(=O)C[C@@H]1C[C@@H](C(=O)N3CC(F)(F)C[C@H]3B(O)O)NC1=O)C2]

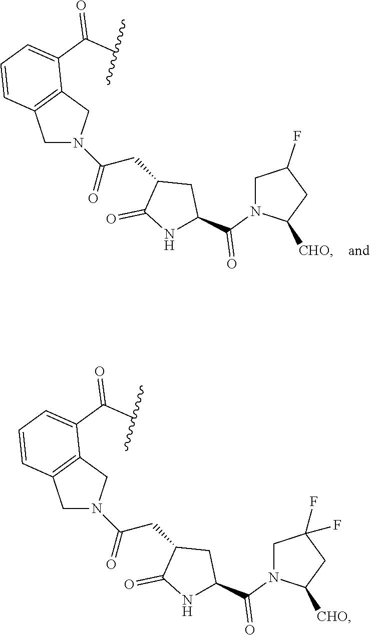 [SMILES: *C(=O)c1cccc2c1CN(C(=O)C[C@@H]1C[C@@H](C(=O)N3CC(F)C[C@H]3C=O)NC1=O)C2 ; "and" ; *C(=O)c1cccc2c1CN(C(=O)C[C@@H]1C[C@@H](C(=O)N3CC(F)(F)C[C@H]3C=O)NC1=O)C2]

5. The conjugate of claim 1, or a pharmaceutically acceptable salt thereof, wherein FL has a structure selected from the group consisting of

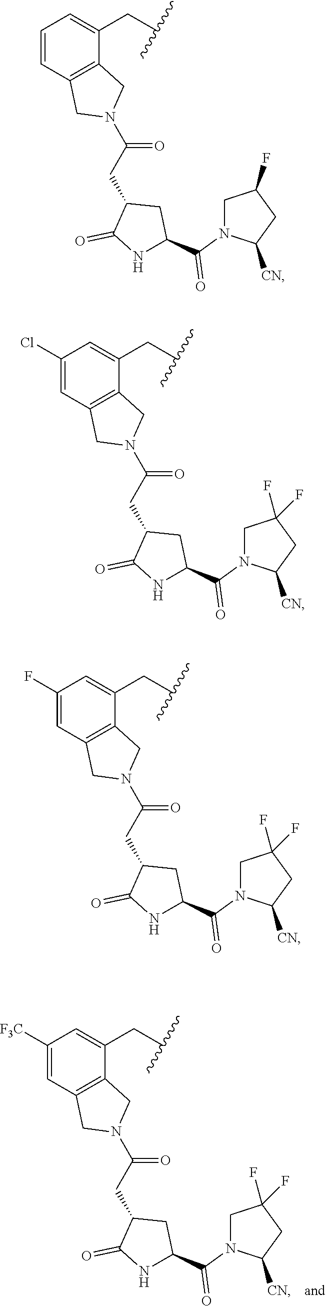 [SMILES: *Cc1cccc2c1CN(C(=O)C[C@@H]1C[C@@H](C(=O)N3C[C@@H](F)C[C@H]3C#N)NC1=O)C2 ; *Cc1cc(Cl)cc2c1CN(C(=O)C[C@@H]1C[C@@H](C(=O)N3CC(F)(F)C[C@H]3C#N)NC1=O)C2 ; *Cc1cc(F)cc2c1CN(C(=O)C[C@@H]1C[C@@H](C(=O)N3CC(F)(F)C[C@H]3C#N)NC1=O)C2 ; *Cc1cc(C(F)(F)F)cc2c1CN(C(=O)C[C@@H]1C[C@@H](C(=O)N3CC(F)(F)C[C@H]3C#N)NC1=O)C2]

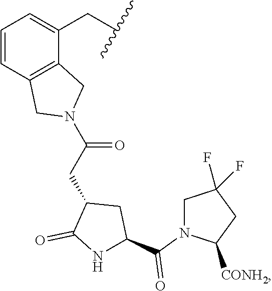 [SMILES: *Cc1cccc2c1CN(C(=O)C[C@@H]1C[C@@H](C(=O)N3CC(F)(F)C[C@H]3C(N)=O)NC1=O)C2]

6. The conjugate of claim 1, or a pharmaceutically acceptable salt thereof, wherein L comprises one or more linker groups independently selected from the group consisting of alkyl(ene), heteroalkyl(ene), heterocycloalkyl(ene), heteroaryl, aryl, alkoxy, thioether, disulfide, carboxylic acid, anhydride, carbonate, carbamate, thioether, sugar, and peptide.

7. The conjugate of claim 1, or a pharmaceutically acceptable salt thereof, wherein L comprises one or more linker groups independently selected from the group consisting of polyethylene glycol (PEG), alkyl(ene), amide, carboxylic acid, anhydride, carbonate, ester, carbamate, thioether, phenyl, and triazole.

8. The conjugate of claim 1, or a pharmaceutically acceptable salt thereof, wherein IA is (CM), in which M is gadolinium (Gd³⁺), manganese (Mn²⁺), or dysprosium (Dy³⁺) and C is a chelator.

9. The conjugate of claim 8, or a pharmaceutically acceptable salt thereof, wherein each C is independently selected from the group consisting of DOTA (1,4,7,10-tetraazacyclododecane-1,4,7,10-tetraacetic acid), TETA (1,4,8,11-tetraazacyclotetradecane-1,4,8,11-tetraacetic acid), SarAr (1-N-(4-Aminobenzyl)-3,6,10,13,16,19-hexaazabicyclo[6.6.6]-eicosane-1,8-diamine), NOTA (1,4,7-triazacyclononane-1,4,7-triacetic acid), NETA (4-[2-(bis-carboxymethylamino)-ethyl]-7-carboxymethyl-[1,4,7]triazonan-1-yl) acetic acid, DTPA (diethylenetriaminepentaacetic acid), or a derivative of any of the foregoing.

10. The conjugate of claim 6, or a pharmaceutically acceptable salt thereof, wherein IA is (CM), in which M is a metal (e.g., gadolinium (Gd³⁺), manganese (Mn²⁺), or dysprosium (Dy³⁺) and C is a chelator.

11. The conjugate of claim 10, or a pharmaceutically acceptable salt thereof, wherein C is selected from the group consisting of DOTA (1,4,7,10-tetraazacyclododecane-1,4,7,10-tetraacetic acid), TETA (1,4,8,11-tetraazacyclotetradecane-1,4,8,11-tetraacetic acid), SarAr (1-N-(4-aminobenzyl)-3,6,10,13,16,19-hexaazabicyclo[6.6.6]-eicosane-1,8-diamine), NOTA (1,4,7-triazacyclononane-1,4,7-triacetic acid), NETA (4-[2-(bis-carboxymethylamino)-ethyl]-7-carboxymethyl-[1,4,7]triazonan-1-yl) acetic acid, DTPA (diethylenetriaminepentaacetic acid), or a derivative of any of the foregoing.

12. The conjugate of claim 7, or a pharmaceutically acceptable salt thereof, wherein IA is (CM), in which M is a metal gadolinium (Gd³⁺), manganese (Mn²⁺), or dysprosium (Dy³⁺) and C is a chelator.

13. The conjugate of claim 12, or a pharmaceutically acceptable salt thereof, wherein C is independently selected from the group consisting of DOTA (1,4,7,10-tetraazacyclododecane-1,4,7,10-tetraacetic acid), TETA (1,4,8,11-tetraazacyclotetradecane-1,4,8,11-tetraacetic acid), SarAr (1-N-(4-aminobenzyl)-3,6,10,13,16,19-hexaazabicyclo[6.6.6]-eicosane-1,8-diamine), NOTA (1,4,7-triazacyclononane-1,4,7-triacetic acid), NETA (4-[2-(bis-carboxymethylamino)-ethyl]-7-carboxymethyl-[1,4,7]triazonan-1-yl) acetic acid, DTPA (diethylenetriaminepentaacetic acid), or a derivative of any of the foregoing.

14. The conjugate of claim 8, or a pharmaceutically acceptable salt thereof, wherein C has a structure

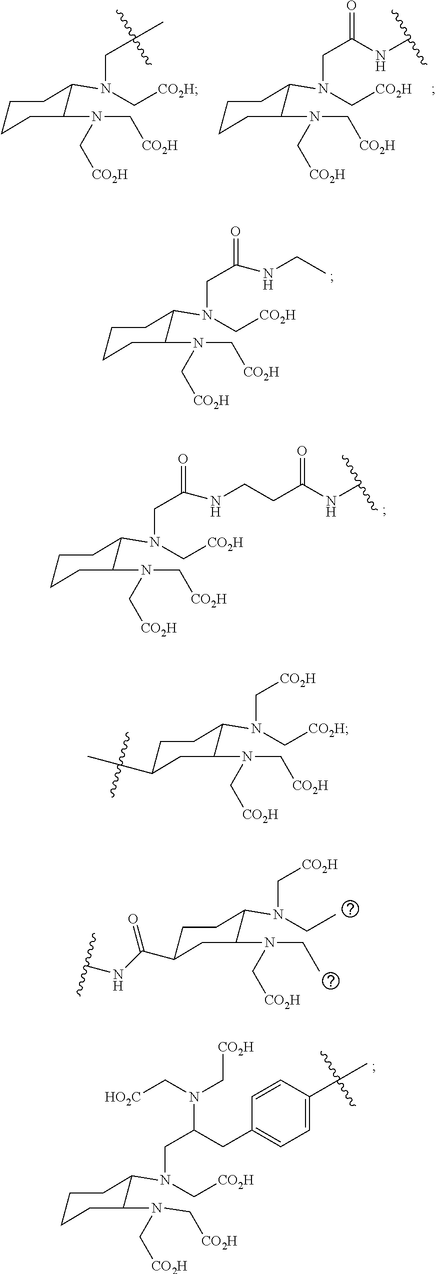

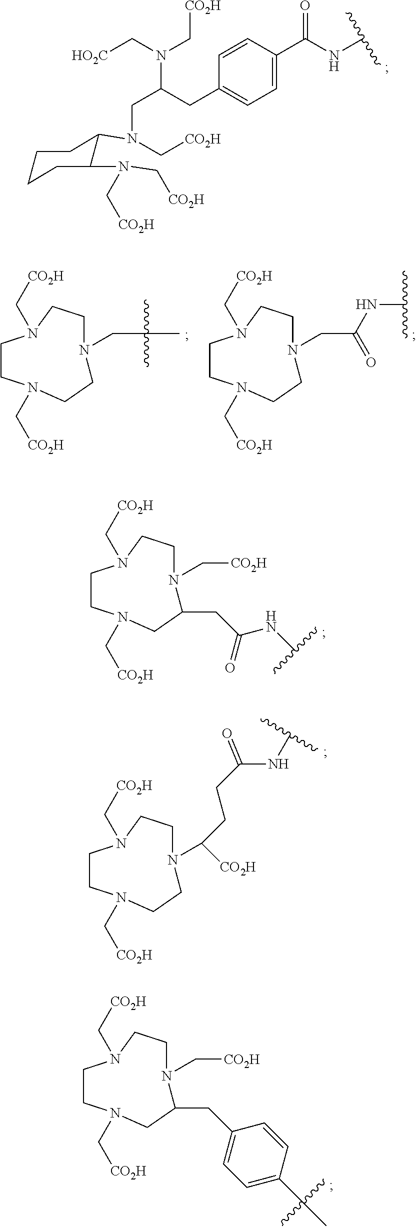 [SMILES: *NC(=O)c1ccc(CC(CN(CC(=O)O)C2CCCCC2N(CC(=O)O)CC(=O)O)N(CC(=O)O)CC(=O)O)cc1 ; *CN1CCN(CC(=O)O)CCN(CC(=O)O)CC1 ; *NC(=O)CN1CCN(CC(=O)O)CCN(CC(=O)O)CC1 ; *NC(=O)CC1CN(CC(=O)O)CCN(CC(=O)O)CCN1CC(=O)O ; *NC(=O)CCC(C(=O)O)N1CCN(CC(=O)O)CCN(CC(=O)O)CC1 ; *c1ccc(CC2CN(CC(=O)O)CCN(CC(=O)O)CCN2CC(=O)O)cc1]

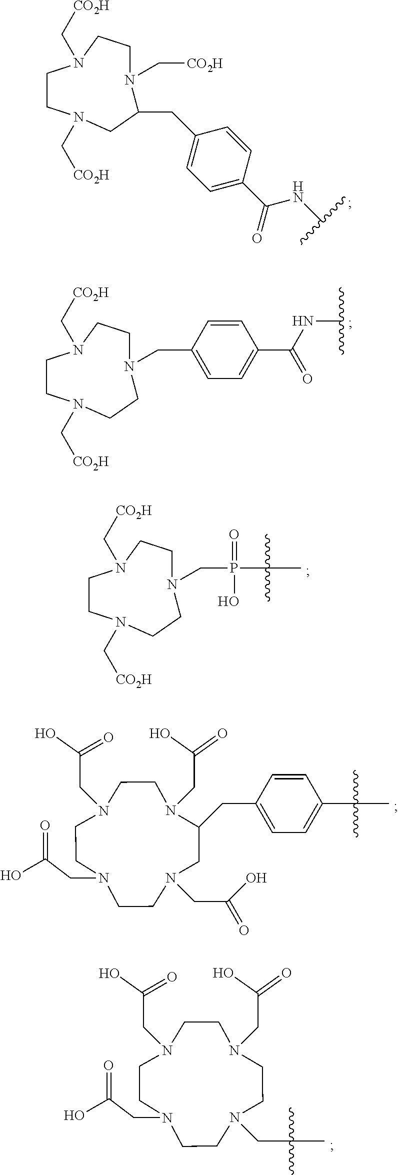

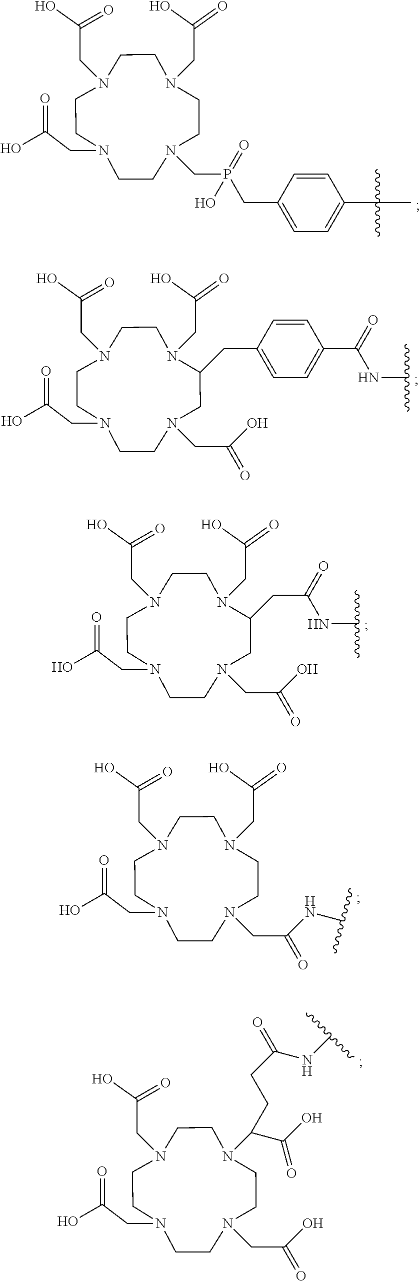 [SMILES: *c1ccc(CP(=O)(O)CN2CCN(CC(=O)O)CCN(CC(=O)O)CCN(CC(=O)O)CC2)cc1 ; *NC(=O)c1ccc(CC2CN(CC(=O)O)CCN(CC(=O)O)CCN(CC(=O)O)CCN2CC(=O)O)cc1 ; *NC(=O)CC1CN(CC(=O)O)CCN(CC(=O)O)CCN(CC(=O)O)CCN1CC(=O)O ; *NC(=O)CN1CCN(CC(=O)O)CCN(CC(=O)O)CCN(CC(=O)O)CC1 ; *NC(=O)CCC(C(=O)O)N1CCN(CC(=O)O)CCN(CC(=O)O)CCN(CC(=O)O)CC1]

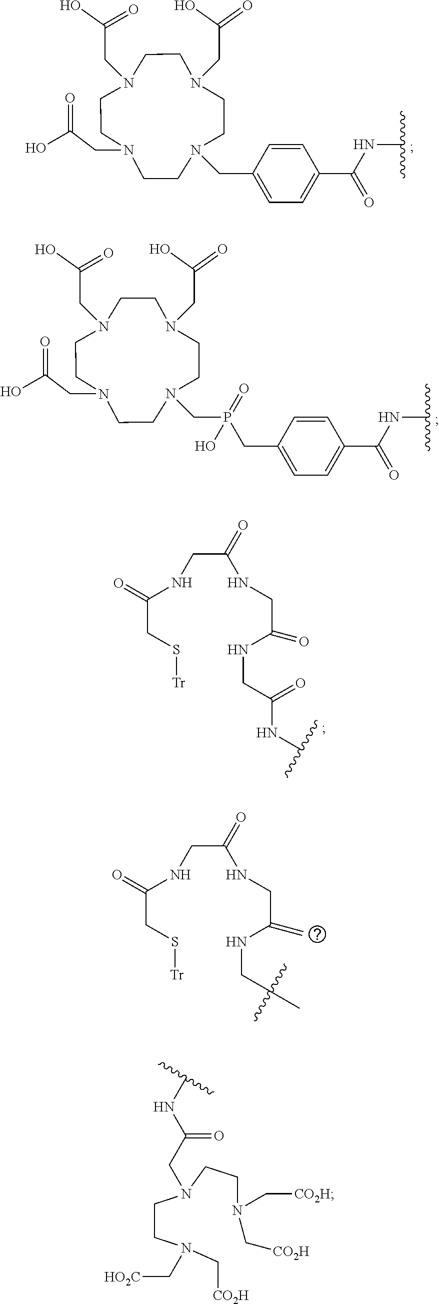

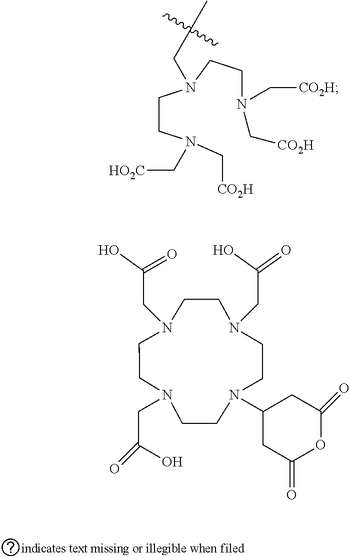 [SMILES: *CN(CCN(CC(=O)O)CC(=O)O)CCN(CC(=O)O)CC(=O)O ; O=C(O)CN1CCN(CC(=O)O)CCN(C2CC(=O)OC(=O)C2)CCN(CC(=O)O)CC1]

15. The conjugate of claim 10, or a pharmaceutically acceptable salt thereof, wherein C has a structure

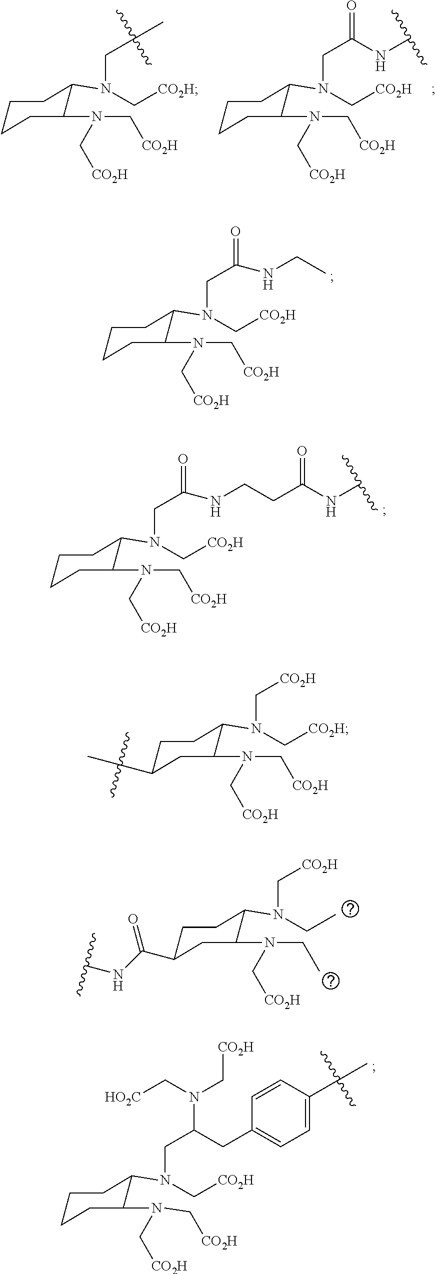

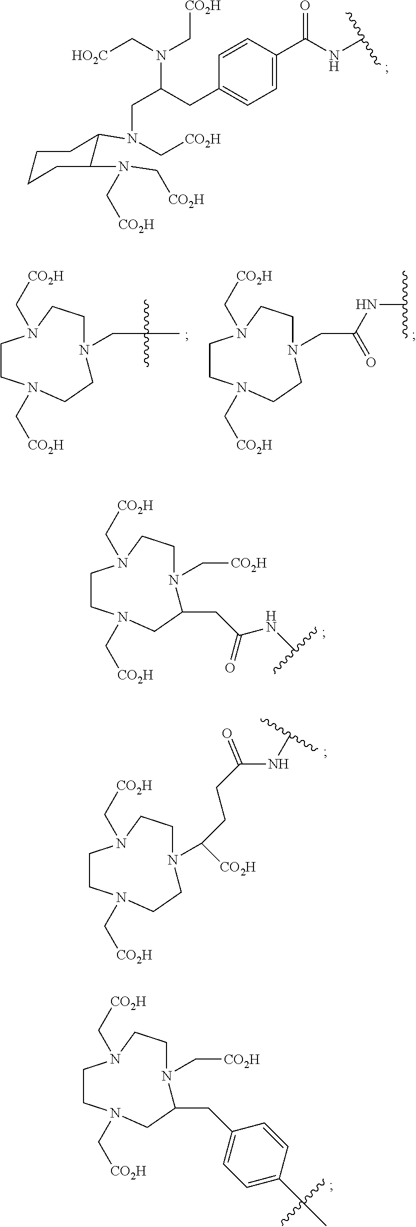 [SMILES: *NC(=O)c1ccc(CC(CN(CC(=O)O)C2CCCCC2N(CC(=O)O)CC(=O)O)N(CC(=O)O)CC(=O)O)cc1 ; *CN1CCN(CC(=O)O)CCN(CC(=O)O)CC1 ; *NC(=O)CN1CCN(CC(=O)O)CCN(CC(=O)O)CC1 ; *NC(=O)CC1CN(CC(=O)O)CCN(CC(=O)O)CCN1CC(=O)O ; *NC(=O)CCC(C(=O)O)N1CCN(CC(=O)O)CCN(CC(=O)O)CC1 ; *c1ccc(CC2CN(CC(=O)O)CCN(CC(=O)O)CCN2CC(=O)O)cc1]

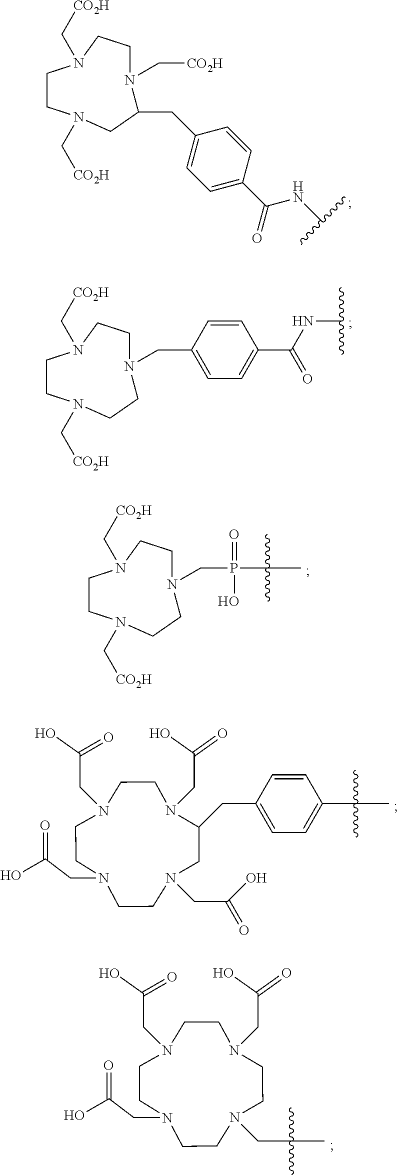

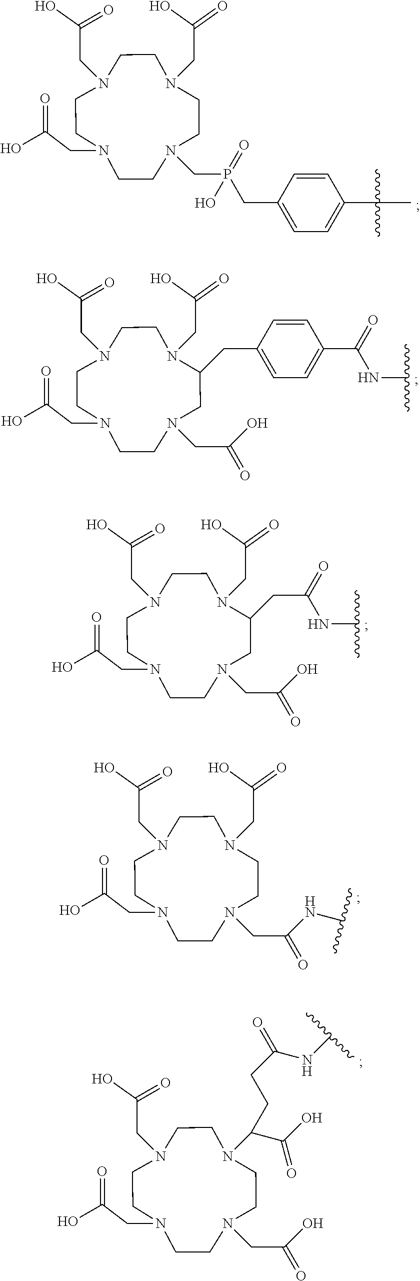 [SMILES: *c1ccc(CP(=O)(O)CN2CCN(CC(=O)O)CCN(CC(=O)O)CCN(CC(=O)O)CC2)cc1 ; *NC(=O)c1ccc(CC2CN(CC(=O)O)CCN(CC(=O)O)CCN(CC(=O)O)CCN2CC(=O)O)cc1 ; *NC(=O)CC1CN(CC(=O)O)CCN(CC(=O)O)CCN(CC(=O)O)CCN1CC(=O)O ; *NC(=O)CN1CCN(CC(=O)O)CCN(CC(=O)O)CCN(CC(=O)O)CC1 ; *NC(=O)CCC(C(=O)O)N1CCN(CC(=O)O)CCN(CC(=O)O)CCN(CC(=O)O)CC1]

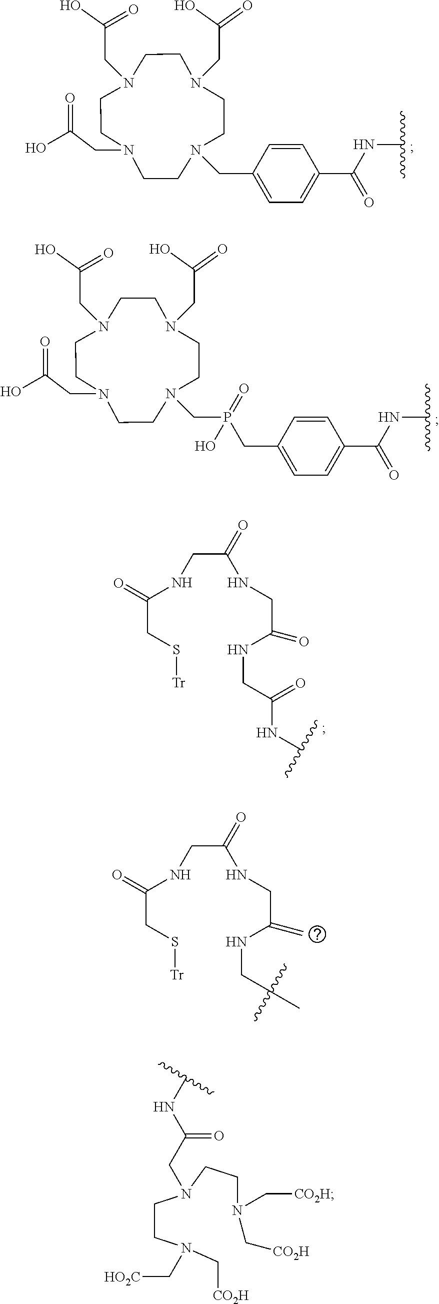

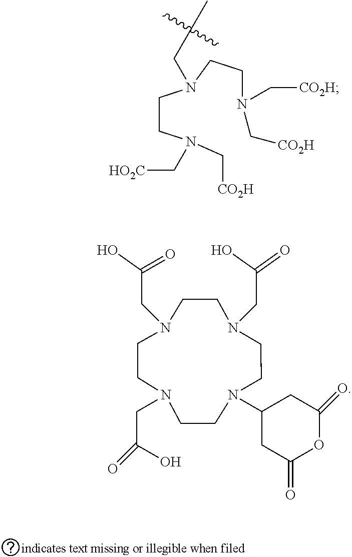 [SMILES: *CN(CCN(CC(=O)O)CC(=O)O)CCN(CC(=O)O)CC(=O)O ; O=C(O)CN1CCN(CC(=O)O)CCN(C2CC(=O)OC(=O)C2)CCN(CC(=O)O)CC1]

16. The conjugate of claim 12, or a pharmaceutically acceptable salt thereof, wherein C has a structure

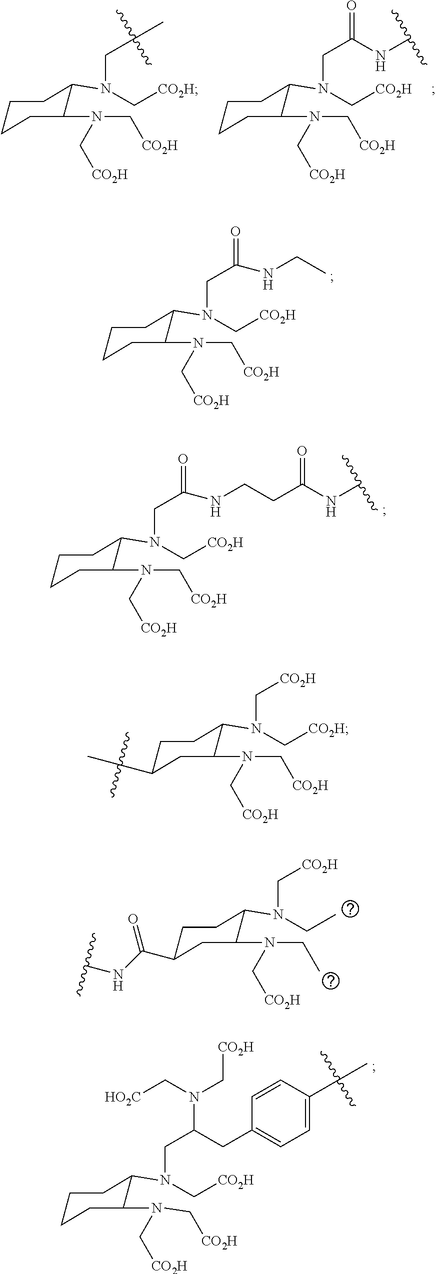

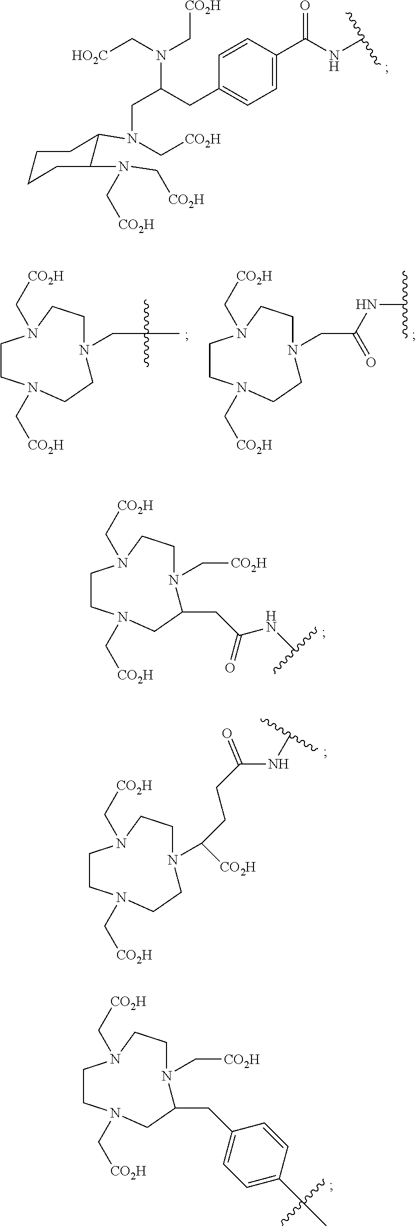 [SMILES: *NC(=O)c1ccc(CC(CN(CC(=O)O)C2CCCCC2N(CC(=O)O)CC(=O)O)N(CC(=O)O)CC(=O)O)cc1 ; *CN1CCN(CC(=O)O)CCN(CC(=O)O)CC1 ; *NC(=O)CN1CCN(CC(=O)O)CCN(CC(=O)O)CC1 ; *NC(=O)CC1CN(CC(=O)O)CCN(CC(=O)O)CCN1CC(=O)O ; *NC(=O)CCC(C(=O)O)N1CCN(CC(=O)O)CCN(CC(=O)O)CC1 ; *c1ccc(CC2CN(CC(=O)O)CCN(CC(=O)O)CCN2CC(=O)O)cc1]

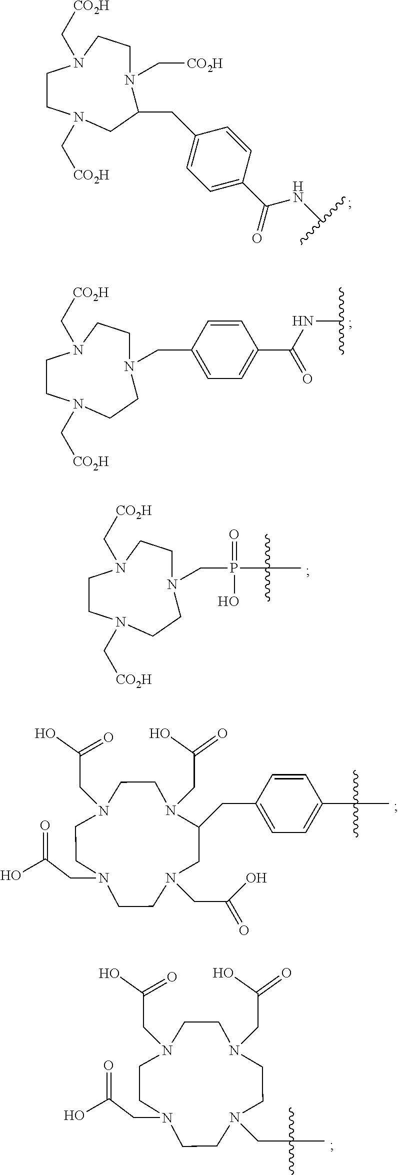

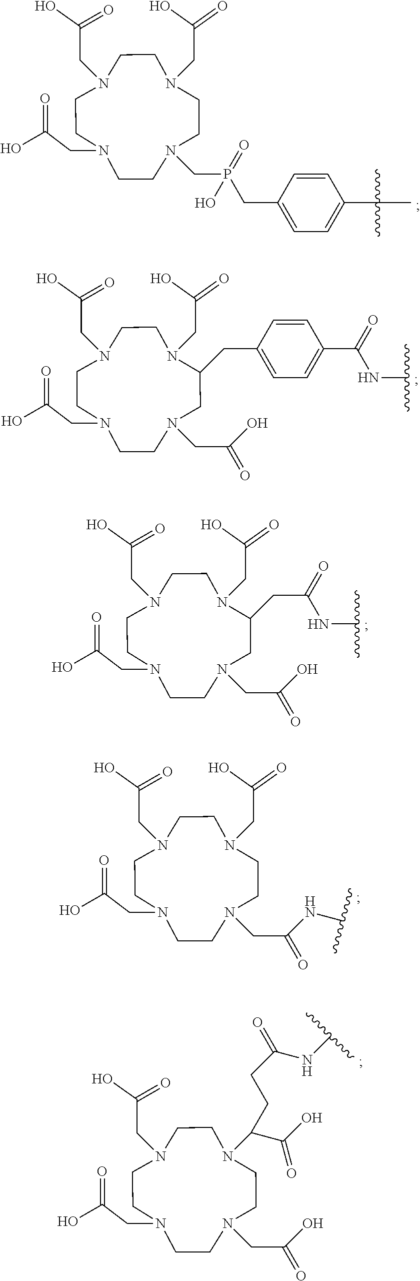 [SMILES: *c1ccc(CP(=O)(O)CN2CCN(CC(=O)O)CCN(CC(=O)O)CCN(CC(=O)O)CC2)cc1 ; *NC(=O)c1ccc(CC2CN(CC(=O)O)CCN(CC(=O)O)CCN(CC(=O)O)CCN2CC(=O)O)cc1 ; *NC(=O)CC1CN(CC(=O)O)CCN(CC(=O)O)CCN(CC(=O)O)CCN1CC(=O)O ; *NC(=O)CN1CCN(CC(=O)O)CCN(CC(=O)O)CCN(CC(=O)O)CC1 ; *NC(=O)CCC(C(=O)O)N1CCN(CC(=O)O)CCN(CC(=O)O)CCN(CC(=O)O)CC1]

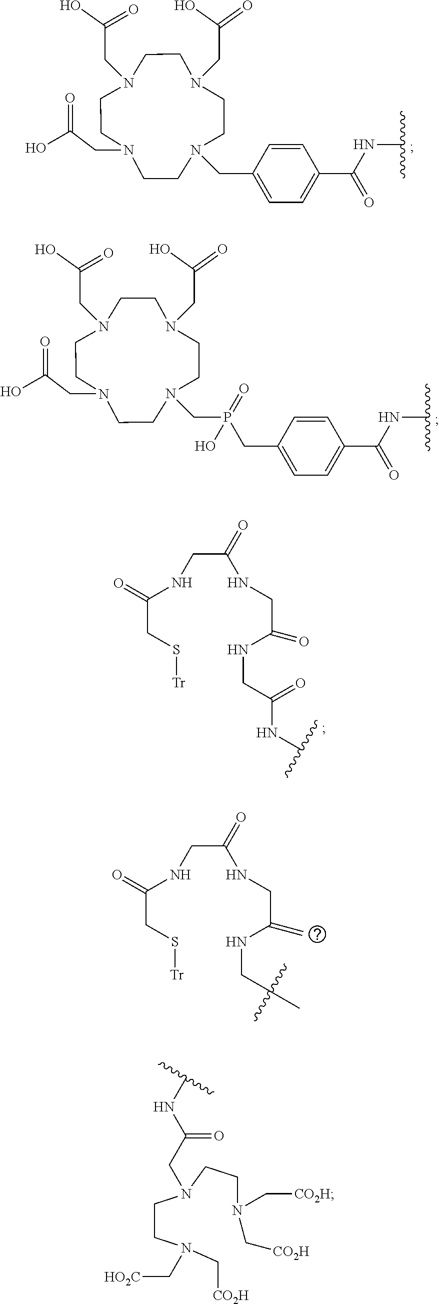

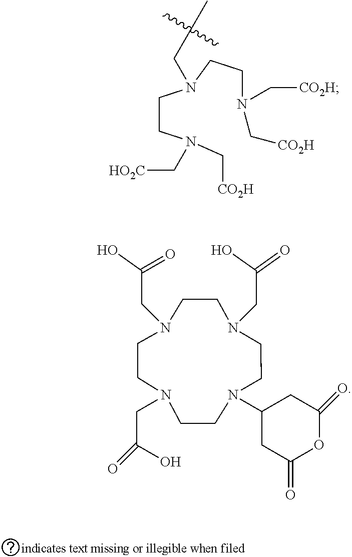 [SMILES: *CN(CCN(CC(=O)O)CC(=O)O)CCN(CC(=O)O)CC(=O)O ; O=C(O)CN1CCN(CC(=O)O)CCN(C2CC(=O)OC(=O)C2)CCN(CC(=O)O)CC1]

17. The conjugate of claim 1, or a pharmaceutically acceptable salt thereof, wherein the MRI agent is a paramagnetic contrast agent.

18. The conjugate of claim 17, or a pharmaceutically acceptable salt thereof, wherein the paramagnetic contrast agent comprises gadolinium (Gd³⁺), manganese (Mn²⁺), or dysprosium (Dy³⁺).

19. The conjugate of claim 18, or a pharmaceutically acceptable salt thereof, wherein the paramagnetic contrast agent is Gd-DOTA (gadoterate dotarem), Gd-HP-DO3A (gadoteridol), Gd-BT-DO3A (gadobutrol), Gd-DTPA (gadopentate dimeglumine), Gd-DTPA-BMEA (gadoversetamide), Gd-DTPA-BMA (gadodiamide), Gd-BOPTA (gadobenate dimeglumine), Gd-EOB-DTPA (gadoxetate), or Ms-325 (gadofosveset).

20. The conjugate of claim 6, or a pharmaceutically acceptable salt thereof, wherein the MRI agent is a paramagnetic contrast agent.

21. The conjugate of claim 20, or a pharmaceutically acceptable salt thereof, wherein the paramagnetic contrast agent comprises gadolinium (Gd³⁺), manganese (Mn²⁺), or dysprosium (Dy³⁺).

22. The conjugate of claim 21, or a pharmaceutically acceptable salt thereof, wherein the paramagnetic contrast agent is Gd-DOTA (gadoterate dotarem), Gd-HP-DO3A (gadoteridol), Gd-BT-DO3A (gadobutrol), Gd-DTPA (gadopentate dimeglumine), Gd-DTPA-BMEA (gadoversetamide), Gd-DTPA-BMA (gadodiamide), Gd-BOPTA (gadobenate dimeglumine), Gd-EOB-DTPA (gadoxetate), or Ms-325 (gadofosveset).

23. The conjugate of claim 7, or a pharmaceutically acceptable salt thereof, wherein the MRI agent is a paramagnetic contrast agent.

24. The conjugate of claim 23, or a pharmaceutically acceptable salt thereof, wherein the paramagnetic contrast agent comprises gadolinium (Gd³⁺), manganese (Mn²⁺), or dysprosium (Dy³⁺).

25. The conjugate of claim 24, or a pharmaceutically acceptable salt thereof, wherein the paramagnetic contrast agent is Gd-DOTA (gadoterate dotarem), Gd-HP-DO3A (gadoteridol), Gd-BT-DO3A (gadobutrol), Gd-DTPA (gadopentate dimeglumine), Gd-DTPA-BMEA (gadoversetamide), Gd-DTPA-BMA (gadodiamide), Gd-BOPTA (gadobenate dimeglumine), Gd-EOB-DTPA (gadoxetate), Ms-325 (gadofosveset), or Mn-DPDP (Mn-dipyridoxyl diphosphate).

26. A pharmaceutical composition comprising a conjugate of claim 1, or a pharmaceutically acceptable salt thereof, and a pharmaceutically acceptable carrier.

27. A method of imaging cells, a tissue, or an organ, any of which express(es) fibroblast activation protein, in a subject, which method comprises administering to the subject a conjugate of claim 1, or a pharmaceutically acceptable salt thereof, or a pharmaceutical composition comprising the conjugate and a pharmaceutically acceptable carrier, and having the cells, the tissue, or the organ in the subject imaged with magnetic resonance imaging.
